SEQUENCE LISTING (24 sequences)

SEQ ID NO: 1 - DNA, 20 bp, Artificial Sequence
  Description of Artificial Sequence Synthetic primer
gacaccactggagggtgact

SEQ ID NO: 2 - DNA, 20 bp, Artificial Sequence
  Description of Artificial Sequence Synthetic primer
caggtccacatggtcttcct

SEQ ID NO: 3 - DNA, 20 bp, Artificial Sequence
  Description of Artificial Sequence Synthetic primer
ctgttcaggcttctcccaag

SEQ ID NO: 4 - DNA, 20 bp, Artificial Sequence
  Description of Artificial Sequence Synthetic primer
tcgttctgcccaagagaagt

SEQ ID NO: 5 - DNA, 20 bp, Artificial Sequence
  Description of Artificial Sequence Synthetic primer
gacatggaccctgagacctg

SEQ ID NO: 6 - DNA, 20 bp, Artificial Sequence
  Description of Artificial Sequence Synthetic primer
gtcattcctccaaggtcagc

SEQ ID NO: 7 - DNA, 20 bp, Artificial Sequence
  Description of Artificial Sequence Synthetic primer
agacctgcccctgcccttct

SEQ ID NO: 8 - DNA, 20 bp, Artificial Sequence
  Description of Artificial Sequence Synthetic primer
ccacacggaggggtgccttc

SEQ ID NO: 9 - DNA, 23 bp, Artificial Sequence
  Description of Artificial Sequence Synthetic primer
acgaaactaccttcaactccatc

SEQ ID NO: 10 - DNA, 23 bp, Artificial Sequence
  Description of Artificial Sequence Synthetic primer
tggtctcaagtcagtgtacaggt

SEQ ID NO: 11 - DNA, 19 bp, Artificial Sequence
  Description of Artificial Sequence Synthetic primer
tgcagaccttcagccagag

SEQ ID NO: 12 - DNA, 20 bp, Artificial Sequence
  Description of Artificial Sequence Synthetic primer
gcttttctgggaaccagtga

SEQ ID NO: 13 - DNA, 18 bp, Artificial Sequence
  Description of Artificial Sequence Synthetic primer
ctgccgctcgagtcactt

SEQ ID NO: 14 - DNA, 18 bp, Artificial Sequence
  Description of Artificial Sequence Synthetic primer
ccccctgccagtatagcc

SEQ ID NO: 15 - DNA, 20 bp, Artificial Sequence
  Description of Artificial Sequence Synthetic primer
ccaagaggaagccctaatcc

SEQ ID NO: 16 - DNA, 20 bp, Artificial Sequence
  Description of Artificial Sequence Synthetic primer
aagatgtagagcgggccttt

SEQ ID NO: 17 - DNA, 20 bp, Artificial Sequence
  Description of Artificial Sequence Synthetic primer
attctgcgcagctttaagga

SEQ ID NO: 18 - DNA, 20 bp, Artificial Sequence
  Description of Artificial Sequence Synthetic primer
gaggtgcccatgctacattt

SEQ ID NO: 19 - DNA, 20 bp, Artificial Sequence
  Description of Artificial Sequence Synthetic primer
ctcttccagccttccttcct

SEQ ID NO: 20 - DNA, 20 bp, Artificial Sequence
  Description of Artificial Sequence Synthetic primer
agcactgtgttggcgtacag

SEQ ID NO: 21 - DNA, 20 bp, Artificial Sequence
  Description of Artificial Sequence Synthetic primer
ccctgcggagtgtgagaagt

SEQ ID NO: 22 - DNA, 20 bp, Artificial Sequence
  Description of Artificial Sequence Synthetic primer
tgcttctgcctcagctgcct

SEQ ID NO: 23 - DNA, 20 bp, Artificial Sequence
  Description of Artificial Sequence Synthetic primer
tggattggacctcaatgaca

SEQ ID NO: 24 - DNA, 20 bp, Artificial Sequence
  Description of Artificial Sequence Synthetic primer
actgtggtccccagaaaatg

(19) United States
(12) Patent Application Publication
(10) Pub. No.: US 2007/0292351 A1
(43) Pub. Date: Dec. 20, 2007

(54) Assay for efficacy of histone deacetylase inhibitors

(75) Inventors: Zuomei Li, Kirkland (CA); Christiane Maroun, Ville-Mon Royal (CA); Jianhong Liu, Montreal (CA); Jeffrey Besterman, Baie D'Urfe (CA); Claire Bonfils, Montreal (CA)

Correspondence Address: KEOWN & ZUCCHERO, LLP, 500 WEST CUMMINGS PARK, SUITE 1200, WOBURN, MA, 01801, US

(73) Assignee: Methylgene, Inc.

(21) Appl. No.: 11/807,206

(22) Filed: May 25, 2007

Related U.S. Application Data
(60) Provisional application No. 60/803,277, filed on May 26, 2006.

(51) Int. Cl. A61K 49/00; C12Q 1/02; C12Q 1/68

(52) U.S. Cl. 424/9.2; 435/29; 435/6

(57) ABSTRACT

The invention provides methods for assessing the efficacy of histone deacetylase inhibitors using biomarkers which can be used in human clinical trials and which are more quantitative, easy to be used and more relevant to clinical outcome for PD monitoring than existing assays. The method according to the invention utilizes biomarkers from blood cells from patients treated with HDAC inhibitors which are easy to assay.

This application claims the benefit of prior U.S. Provisional Application Ser. No. 60/803,277, filed on May 26, 2006, the entire teachings of which are incorporated herein by reference.

### BACKGROUND OF THE INVENTION

1. Field of the Invention

The invention relates to histone deacetylase inhibitors. More particularly, the invention relates to methods for assessing the efficacy of histone deacetylase inhibitors using biomarkers as surrogates for efficacy.

2. Summary of the Related Art

HDAC inhibitors have emerged as novel agents for multiple human diseases, including cancer, neurodegenerative diseases, psychiatric disorders, inflammation and autoimmune diseases as well as metabolic diseases. Currently multiple cancer clinical trials using structurally distinct HDAC inhibitors have been initiated.

Analysis of pharmacodynamic (PD) properties of these HDAC inhibitors is important not only for understanding drug exposure but also important to reveal molecular parameters which can be used to predict the clinical outcome. To date most of the PD characterizations in clinical trials are focused on measuring core histone acetylation in peripheral white cells from patients. However, methods to detect histone acetylation depend on fairly large amounts of cells which can be limiting in patients and often variability in these assays has been observed. More importantly, it is still unclear whether there is any dose-dependent correlation between the increase in levels of histone acetylation with the drug exposure or with the clinical efficacy. In cells, histone deacetylases also directly deacetylate transcription factors, in addition to histones, to regulate transcription. Therefore histone acetylation may not be the most relevant biomarker to guide clinical trials for HDAC inhibitors.

An increasingly growing body of literature describes genes regulated by HDAC inhibitors in in vitro settings. These include induction of cell cycle inhibitors such as the cyclin-dependent kinase inhibitor p21, induction of proapoptotic proteins such as caspases-3 and 9 and also Bax and Trail ligand, a member of the TNF superfamily, as well as downregulation of angiogenesis factors such as the VEGF and hypoxia-inducible factor (HIF). In peripheral blast cells from AML patients, MS-275 has also been demonstrated to increase the level of the pro-apoptotic TRAIL expression upon ex vivo treatment (Nebbioso A et. al. Nature Medicine December 2004). Reports describing the biomarkers of HDAC inhibitors in vivo are sparse and come mainly from studies using a pan-inhibitor, FK228 (Graham, C. et al Clin. Cancer Res. 12: 224; Sasakawa T et al Biochem Pharmacol. 2005 69(4):603-16). Unfortunately, these studies describe only biomarkers in tumor tissues. There is a need to develop other biomarkers which can be used in human clinical trials which are more quantitative, easy to be used and more relevant to clinical outcome for PD monitoring. Preferrably, biomarkers from blood cells from patients treated with HDAC inhibitors should be used as they are easy to assay.

### BRIEF SUMMARY OF THE INVENTION

The invention provides methods for assessing the efficacy of histone deacetylase inhibitors using biomarkers which can be used in human clinical trials and which are more quantitative, easy to be used and more relevant to clinical outcome for PD monitoring than existing assays. The method according to the invention preferably utilizes biomarkers from blood cells from patients treated with HDAC inhibitors which are easy to assay.

### BRIEF DESCRIPTION OF THE DRAWINGS

FIG. 1. Induction of transcription of MT3 in human colon cancer HCT15 cells in vitro by HDAC inhibitors MGCD0103, MS-275 and SAHA, but not by an inactive analog of MGCD0103 (Compound A) or a CDK inhibitor (See Table 1 for structures). HCT15 cells were treated with HDAC inhibitors or indicated compounds for 16 hours before total RNAs are isolated. RNA levels of MT3 were measured by real-time RT-PCR and are subsequently normalized against β-actin.

FIG. 2. Dose-dependent induction of MT3 transcription by MGCD0103 in vitro in various human cancer cell lines from different tissue origins, include colon cancer HCT15 cells, Jurkat-T leukemic cells or RPMI-8226 myeloma cells. Cells were treated with MGCD0103 or its inactive analog (Compound A) at indicated doses for 16 h, then RNA was extracted and the level of MT3 was measured by conventional semi-quantitative RT-PCR.

FIG. 3 Time-dependent induction of MT3 transcription in HCT15 cells by MGCD0103 in vitro. Colon cancer HCT15 cells were exposed to 1 μM MGCD0103 for various amounts of time, and MT3 RNA levels were monitored by conventional semi-quantitative RT-PCR. Maximal induction is achieved between 8 h and 24 h of exposure and maintained through 72 h.

FIG. 4 Synergistic induction of MT3 transcription by MGCD0103 and a demethylating agent (5-aza-deoxyC) in human gastric carcinoma MKN45 cells in vitro. Cells were treated with 0.5 μM 5-azadC either alone, or in combination with MGCD0103 at indicated doses. When applicable, 5-azadC exposure was for a total of 96 h. When MGCD0103 was used, it was only added in the last 24 h of the schedule.

FIG. 5 Induction of MT3 and p21 in implanted human NSCLC H460 tumors in vivo in nude mice. Nude mice (Balb/c) bearing human H460 tumors were treated with 100 mg/kg MGCD0103 or 0.5% HEC (Vehicle) by oral administration. After 6 h or 24 h, three mice from each group were sacrificed, their tumors harvested and analyzed. Panel A: p21 and MT3 RNA levels quantified by conventional semi-quantitative PCR, normalized to β-actin. Panel B: H4Ac level detected by immunoblot analysis, normalized to total histones. H4 acetylation is detectable only after 6 h of treatment, but gene induction persists until at least 24 h.

FIG. 6 Induction of MT3 transcription ex vivo in both time-dependent and dose-dependent manner. Peripheral white blood cells were isolated from healthy volunteers and treated ex vivo with MGCD0103 at indicated doses for 24 hr or 48 hr. RNA was extracted to monitor MT3 mRNA level by conventional semi-quantitative PCR (normalized to β-actin).

FIG. 7 Dose-dependent HDAC inhibition in vivo in patients with solid tumor who received treatment with MGCD0103 for one week (three doses per week). At day 8 (72 h after the third dose), peripheral white blood cells were tested for HDAC activity by whole cell assay

FIG. 8. Induction of MT3 (A) and AREG (B) transcription in vivo in patients with solid tumor who received treatment with MGCD0103 for one week (three doses per week). At day 8 (72 h after the third dose), peripheral white blood cells were isolated and RNA extracted and analyzed by qRT-PCR

FIG. 9 Time-dependent inhibition of HDAC activity and induction of histone H3 acetylation in peripheral white cells from an AML patient (Patient A) which achieves clinical response after MGCD0103 treatment in vivo.

FIG. 10. Reduction of bone marrow blast count in Patient A after 2 cycles of MGCD0103 treatment in vivo.

FIG. 11 Induction of transcription of a subset of proapoptotic proteins in peripheral blast cells (sample from D8 vs 0 h) from an AML patient with response (Patient A) comparing to that of another three AML patients with no responses (Patient B, C, D) in the same dose range, as determined by microarray expression analysis.

FIG. 12. Induction of transcription of three tumor suppressor genes, BTG1, TNFSF9 and p21 in peripheral white cells from Patient A in vivo as determined by real time RT-PCR. Note that both BTG1 and TNFSF9 were identified in FIG. 11 using microarray expression analysis and their expression is confirmed in vivo by real time RT-PCR.

FIG. 13. Induction of transcription of three tumor suppressor genes, BTG1, TNFSF9 and p21 in peripheral white cells from a MDS patient with clinical response (Patient E) and a MDS patient without response (Patient F) in vivo as determined by real time RT-PCR. Blood samples were drawn 48 hr post the 2^{nd} dose in cycle 1.

FIG. 14 Dose-dependent induction of IL-6 transcription in human leukemia RPMI8226 and Jurkat cells treated by MGCD0103 but not its inactive analog Compound A. Cells were treated 24 hours before RNA extraction.

FIG. 15 Induction of transcription of IL-6, IL-8, IL-1b, MIP1b cytokine/chemokines by MGCD0103 in peripheral white cells ex vivo from a MDS patient and in vivo in an AML patient with response (Patient J). No such induction was observed in peripheral white cells from healthy volunteers ex vivo treated with MGCD0103 or two other AML patients without clinical response (Patient B, C) treated with MGCD0103 in vivo. Induction of IL-6/IL-8/IL-1b/MIP1b by MGCD0103 ex vivo/in vivo in patients is specific as the expression level of other cytokine/chemokines is either decreased or remained unmodified.

FIG. 16 Induction of IL-6 and IL-8 expression in plasma from two leukemia patients treated with MGCD0103 orally in vivo, as determined by cytokine antibody array.

FIG. 17 Induction of plasma IL-6 from leukemia patients from treated with MGCD0103 at Day 8, analyzed by ELISA assays.

FIG. 18 Dose-dependent induction of IL-6 in plasma from AML/MDS patients by combination treatment of Vidaza and MGCD0103. Patient plasma samples at day 21 were analyzed by ELISA

### DETAILED DESCRIPTION OF THE PREFERRED EMBODIMENTS

The invention provides methods for assessing the efficacy of histone deacetylase inhibitors using biomarkers which can be used in human clinical trials and which are more quantitative, easy to be used and more relevant to clinical outcome for PD monitoring than existing assays. The present invention is useful in a multiple of human diseases, including but not limited to, cancer, neurodegenerative diseases, psychiatric disorders, inflammation and autoimmune diseases as well as metabolic diseases. In some preferred embodiments, “efficacy” denotes the ability of the histone deacetylase inhibitor to inhibit the growth of cancer cells in a mammal, preferably a human cancer patient. Such cancer cells may be present in a solid tumor or a diffuse cancer such as leukemia. In another preferred embodiment, “efficacy” denotes the ability of the histone deacetylase inhibitor to inhibit inflammatory diseases. The present invention also provides methods for prescreening a drug candidate, for example in an animal model, to determine if it would be active in an in vivo system prior to clinical testing. The method according to the invention preferably utilizes biomarkers from blood cells from patients treated with HDAC inhibitors which are easy to assay.

The references cited herein reflect the level of knowledge in the field and are hereby incorporated by reference in their entirety. Any conflicts between the teachings of the cited references and this specification shall be resolved in favor of the latter.

In a first aspect, the invention provides a method for assessing the efficacy of a histone deacetylase inhibitor alone or in conjuction with an other agent in a mammal comprising obtaining peripheral blood cells from a mammal that has not been treated with the histone deacetylase inhibitor (or with the histone deacetylase and other agent); determining a level of expression in the peripheral blood cells of a set of at least one or more genes or gene products thereof selected from the group consisting of a cell cycle blocking gene, a cell cycle blocking gene product, an apoptosis gene, an apoptosis gene product, a preapoptosis gene, a preapoptosis gene product, an anti-proliferation gene, an anti-proliferation gene product, an anti-angiogenesis gene and an anti-angiogenesis gene product, a differentiation induction gene, a differentiation induction gene product, a gene encoding antitumor soluble factors, an antitumor soluble factor, a gene encoding transcriptional factor, a transcriptional factor, a gene encoding soluble factor, a soluble factor; treating the mammal with the histone deacetylase inhibitor (or with the histone deacetylase and other agent); obtaining peripheral blood cells from the mammal treated with the histone deacetylase inhibitor (or with the histone deacetylase and other agent); determining the level of expression in the peripheral blood cells from the mammal treated with the histone deacetylase inhibitor (or with the histone deacetylase and other agent) of the same set of at least one or more genes; and comparing the level of expression of the set of the at least one or more genes from the peripheral blood cells of the mammal that has not been treated with the histone deacetylase inhibitor (or with the histone deacetylase and other agent) against the level of expression of the set of at least one or more genes from the peripheral blood cells of the mammal after it has been treated with the histone deacetylase inhibitor (or with the histone deacetylase and other agent), wherein increased expression of the set of at least one or more genes from the peripheral blood cells of the mammal after it has been treated with the histone deacetylase inhibitor (or with the histone deacetylase and other agent) relative to the level of expression of the set of the at least one or more genes from the peripheral blood cells of the mammal that has not been treated with the histone deacetylase inhibitor (or with the histone deacetylase and other agent) is indicative of efficacy of the histone deacetylase inhibitor (or with the histone deacetylase and other agent) in the mammal.

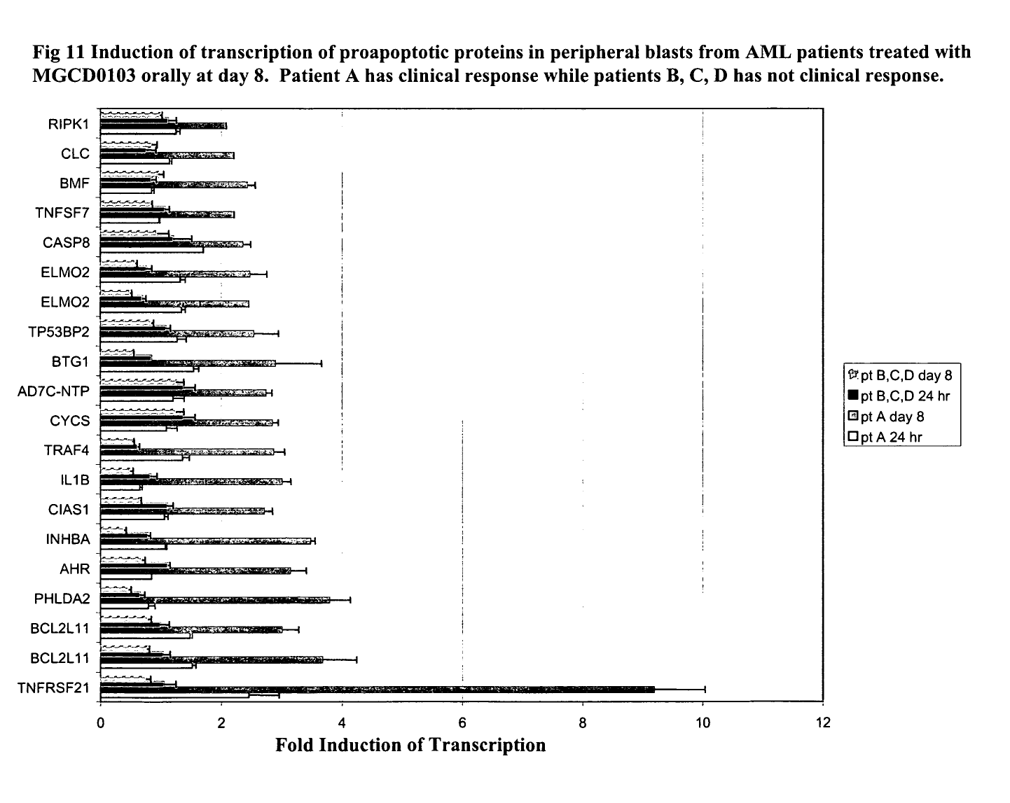

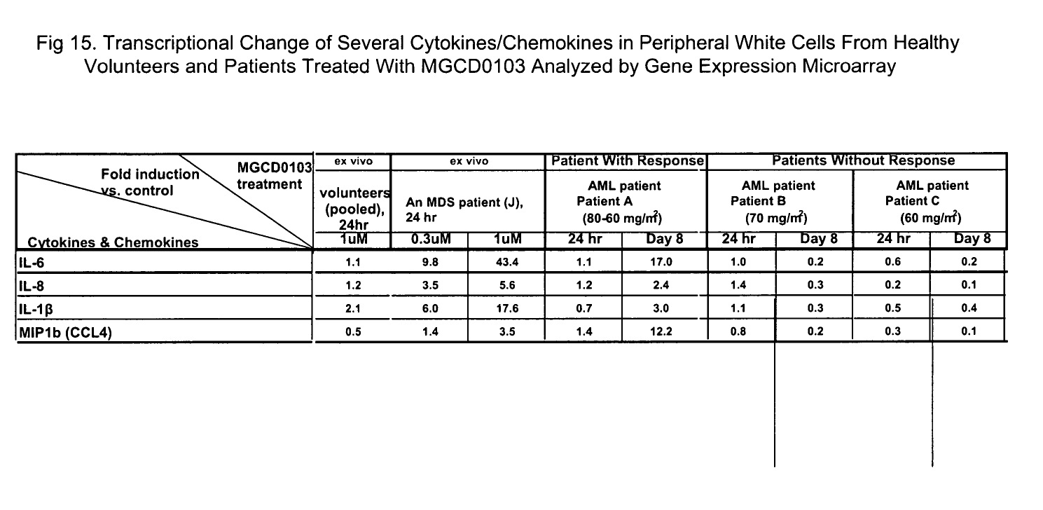

In certain preferred embodiments, the genes or gene products thereof is selected from Table 2 to Table 6 and FIG. 11 and FIG. 15. In certain embodiments, the genes or gene products thereof is selected from the group consisting of FOXO1A, IER3, UNC5B, GADD45β, RGS2, KLF4, TNFSF9, TNFSF15, PDCD1, KLRC1, KLRC4, YPEL4, CDKN1A (P21), GADD45α, GADD45b, BTG1 and MT3, EREG, GDF15, BAI2, AREG, CXCL14, PROM1, CDKN1C, SOD2, SNIP, TNF, KRTHA2, BMF, CD40, TNFSF14, HIPK2, CASP7, IL1B, GPR65, EIF2AK2, BNIP3L, AHR, PRKAR2B, ADORA1, DNASE2, TNFRSF21, LY86, APOE, TNFSF10, AXUD1, IL3RA, NALP1, MX1, CLU, PDE1B, CASP5, CAST, CASP4, TNFRSF25, PPP3CA, MAP3K14, NGFR, CCL7, CCL4 (MIP1b), IFNG, THBS1, BIN1, DUSP4, CXCL1, SEMA6B, NRG1, IL10, APC, CTNNBL1, TNFRSF1A, FOXO3A, CD163, TNFSF14, LASTS2, NRG1, RIPK1, CLC, TNFSF7, CASP8, ELMO2, TP53BP2, AD7C-NTP, CYCS, TRAF4, CIAS1, INHBA, PHLDA2, BCL2L11, IL-6, IL-8

In certain preferred embodiments, the gene or gene product thereof is selected from the group consisting of MT3, TNFSF7, BTG1, IL-6, IL-8, IL1b, CCL4, CCL7, IFNG, THBS1, BIN1, DUSP4, TNFRSF21, CXCL1, SEMA6b, NRG1, IL10, APC, CTNNBL1, TNFRSF1a, FOXO3a, CD163, TNFSF14, LAST2, CXCL14, IER3, PROM1, CDKN1c, SOD2, SNIP, TNF, KRTHA2;

In certain preferred embodiments, the gene or gene product thereof is selected from the group consisting of MT3, TNFSF7, BTG1, IL-6, IL-8, IL1b, CCL4, CCL7, IFNG, THBS1, TNFRSF21, CXCL1, NRG1, IL10, APC, TNFRSF1a, FOXO3a, BMF, ELMO2, BCL2L11;

In certain preferred embodiments, the level of expression is the level of RNA.

In certain preferred embodiments, the level of expression is the level of protein encoded by the one or more genes

In a second aspect, the invention provides a method for assessing the efficacy of a histone deacetylase inhibitor in a mammal comprising obtaining serum from a mammal that has not been treated with the histone deacetylase inhibitor (alone or in conjuction with an other cancer therapeutic), determining a level of a set of at least one or more circulating serum proteins in the serum from the mammal, treating the mammal with the histone deacetylase inhibitor (alone or in conjuction with an other cancer therapeutic), obtaining serum from the mammal treated with the histone deacetylase inhibitor (alone or in conjuction with an other cancer therapeutic), determining the level of the same set of at least one or more proteins in the serum from the mammal treated with the histone deacetylase inhibitor (alone or in conjuction with an other cancer therapeutic), and comparing the level of the set of at least one or more proteins in the serum from the mammal that has not been treated with the histone deacetylase inhibitor (alone or in conjuction with an other cancer therapeutic) against the level of the set of at least one or more proteins in the serum from the mammal after it has been treated with the histone deacetylase inhibitor (alone or in conjuction with an other cancer therapeutic), wherein increased levels of the set of at least one or more proteins in the serum from the mammal after it has been treated with the histone deacetylase inhibitor (alone or in conjuction with an other cancer therapeutic) relative to the level of the set of at least one or more proteins in the serum from the mammal that has not been treated with the histone deacetylase inhibitor (alone or in conjuction with an other cancer therapeutic) is indicative of efficacy of the histone deacetylase inhibitor (alone or in conjuction with an other cancer therapeutic) in the mammal.

In certain preferred embodiments, the circulating serum protein is selected from the group consisting of a cytokine, a chemokine, a soluble receptor, a hormone and an antibody. In certain preferred embodiments, the circulating serum protein is selected from the group consisting of TNFSF9, TNFSF15, EREG, AREG, CXCL14, TNF, TNFSF14, IL1B, CCL7, CCL4 (MIP1b), IFNG, THBS1, CXCL1, IL10, NRG1, TNFSF7, IL-6, IL-8

In a third aspect, the invention provides a method for assessing efficacy of an HDAC inhibitor (alone or in conjuction with an other therapeutic), in a patient comprising obtaining a first sample of cells from the patient, treating the patient with the HDAC inhibitor (alone or in conjuction with an other therapeutic), obtaining a second sample of cells from the patient, assessing the level of expression of one or more genes or gene products from the group consisting of the genes disclosed in Tables 2-6, or gene products thereof, in the first sample of cells and in the second sample of cells, and comparing the level of expression of the one or more genes, or gene products thereof, in the first sample of cells with the level of expression of the one or more genes, or gene products thereof, in the second sample of cells, wherein the HDAC inhibitor is efficacious if the level of expression of the one or more genes, or gene products thereof, in the second sample of cells is greater than the level of expression of the one or more genes, or gene products thereof, in the first sample of cells.

In certain preferred embodiments, the level of expression of the one or more genes is determined by measuring the level of proteins encoded by the one or more genes. In certain preferred embodiments, the level of expression of the one or more genes is determined by measuring the level of RNA expressed from the one or more genes.

In certain preferred embodiments, the one or more genes is selected from the group consisting of FOXO1A, IER3, UNC5B, GADD45p, RGS2, KLF4, TNFSF9, TNFSF15, PDCD1, KLRC1, KLRC4, RYBP, YPEL4, CDKN1A (P21), GADD45b, BTG1 and MT3, EREG, GDF15, BAI2, AREG, CXCL14, PROM1, CDKN1C, SOD2, SNIP, TNF, KRTHA2, BMF, CD40, TNFSF14, HIPK2, CASP7, IL1B, GPR65, EIF2AK2, BNIP3L, AHR, PRKAR2B, ADORA1, DNASE2, TNFRSF21, LY86, APOE, TNFSF10, AXUD1, IL3RA, NALP1, MX1, CLU, PDE1B, CASP5, CAST, CASP4, TNFRSF25, PPP3CA, MAP3K14, NGFR, CCL7, CCL4 (MIP1b), IFNG, THBS1, BIN1, DUSP4, CXCL1, SEMA6B, NRG1, IL10, APC, CTNNBL1, TNFRSF1A, FOXO3A, CD163, TNFSF14, LASTS2, NRG1, RIPK1, CLC, TNFSF7, CASP8, ELMO2, TP53BP2, AD7C-NTP, CYCS, TRAF4, CIAS1, INHBA, PHLDA2, BCL2L11, IL-6, IL-8

In certain preferred embodiments, the gene or gene product thereof is selected from the group consisting of MT3, TNFSF7, BTG1, IL-6, IL-8, IL1b, CCL4, CCL7, IFNG, THBS1, BIN1, DUSP4, TNFRSF21, CXCL1, SEMA6b, NRG1, IL10, APC, CTNNBL1, TNFRSF1a, FOXO3a, CD163, TNFSF14, LAST2, CXCL14, IER3, PROM1, CDKN1c, SOD2, SNIP, TNF, KRTHA2;

In certain preferred embodiments, the gene or gene product thereof is selected from the group consisting of MT3, TNFSF7, BTG1, IL-6, IL-8, IL1b, CCL4, CCL7, IFNG, THBS1, TNFRSF21, CXCL1, NRG1, IL10, APC, TNFRSF1a, FOXO3a, BMF, ELMO2, BCL2L11.

In certain preferred embodiments, the one or more genes is selected from the group consisting of FOXO1A, IER3, UNC5B, GADD45p, RGS2, KLF4, TNFSF9, TNFSF15, KLRC1, KLRC4, YPEL4, CDKN1A (P21), GADD45b, BTG1 and MT3, EREG, GDF15, BAI2, AREG, CXCL14, PROM1, CDKN1C, SOD2, SNIP, TNF, KRTHA2, BMF, CD40, TNFSF14, HIPK2, CASP7, IL1B, GPR65, EIF2AK2, BNIP3L, AHR, PRKAR2B, ADORA1, DNASE2, TNFRSF21, LY86, APOE, TNFSF10, AXUD1, IL3RA, NALP1, MX1, CLU, PDE1B, CASP5, CAST, CASP4, TNFRSF25, PPP3CA, MAP3K14, NGFR, CCL7, CCL4 (MIP1b), IFNG, THBS1, BIN1, DUSP4, CXCL1, SEMA6B, NRG1, IL10, APC, CTNNBL1, TNFRSF1A, FOXO3A, CD163, TNFSF14, LASTS2, NRG1, RIPK1, CLC, TNFSF7, CASP8, ELMO2, TP53BP2, AD7C-NTP, CYCS, TRAF4, CIAS1, INHBA, PHLDA2, BCL2L11, IL-6, IL-8

In certain preferred embodiments, the gene or gene product thereof is selected from the group consisting of MT3, TNFSF7, BTG1, IL-6, IL-8, IL1b, CCL4, CCL7, IFNG, THBS1, BIN1, DUSP4, TNFRSF21, CXCL1, SEMA6b, NRG1, IL10, APC, CTNNBL1, TNFRSF1a, FOXO3a, CD163, TNFSF14, LAST2, CXCL14, IER3, PROM1, CDKN1c, SOD2, SNIP, TNF, KRTHA2;

In certain preferred embodiments, the gene or gene product thereof is selected from the group consisting of MT3, TNFSF7, BTG1, IL-6, IL-8, IL1b, CCL4, CCL7, IFNG, THBS1, TNFRSF21, CXCL1, NRG1, IL10, APC, TNFRSF1a, FOXO3a, BMF, ELMO2, BCL2L11.

In certain preferred embodiments, the level of expression of the on or more genes or gene products thereof in the second sample of cells is at least 2.5-fold greater than the level of expression of the one or more genes or gene products thereof in the first sample of cells.

In certain preferred embodiments, the level of expression is the level of RNA.

In certain preferred embodiments, the level of expression is the level of protein encoded by the one or more genes.

In certain preferred embodiments, the one or more genes is selected from the group consisting of FOXO1A, IER3, UNC5B, GADD45β, RGS2, KLF4, IL-18, TNFSF9, DDIT4, SMARCD3, PDCD1, KLRC1, KLRC4, RYBP, YPEL4, CARD10, ZFP36, BCL6, p21, GADD45α, BTG1 and MT3.

In certain preferred embodiments, the one or more genes comprises IL-18, TNFSF9, IL-6 or IL-8.

In certain preferred embodiments, the gene or gene product thereof is selected from the group consisting of MT3, p21, AREG, BTG1, TNFSF9, IL-6, IL-8, IL-1b and MIP1b cytokines/chemokines.

In each of the above methods according to the invention, the cells can be from a variety of sampling sources. In certain preferred embodiments, the cells are peripheral blood cells. In certain preferred embodiments, the cells are blast cells. In certain preferred embodiments, the cells are tumor cells. In certain preferred embodiments, the cells are cells from skin biopsy. In certain preferred embodiments, the cells are cells from buccal swipe.

In another aspect, the invention provides a method for screening a compound for HDAC inhibitory activity, comprising: a) administering a compound to cells to obtain treated cells; b) assaying for expression levels of a set of at least one or more genes selected from the group consisting of those disclosed in any of Tables 2-6 and in FIG. 11 and FIG. 15 in the treated cells and in control cells to which no compound has been administered; and c) comparing the expression levels between the treated cells and the control cells wherein a difference in the expression levels between the treated cells and control levels indicates whether the compound possesses HDAC inhibitor activity.

In certain preferred embodiments, the expression levels is the level of RNA.

In certain preferred embodiments, the expression level is the level of protein encoded by the one or more genes.

In certain preferred embodiments, the cells are selected from the group consisting of a blast cell, a blood cell, a tumor cell line and a tumor cell.

In certain preferred embodiments, the cells are in vivo.

In certain preferred embodiments, the cells are in vitro.

In another aspect, the invention provides a method for determining the sensitivity of a cell to a histone deacetylase inhibitor comprising: a) administering the histone deacetylase inhibitor to the cell; b) determining a level of expression of a set of at least one or more genes or gene products thereof selected from the group disclosed in any of Tables 2-6 and FIG. 11 and FIG. 15 in or by the cells and in or by control cells to which no histone deacetylase inhibitor has been administered; and c) comparing the levels of expression between the cells and the control cells, wherein a difference in levels of expression of the set of at least one or more genes or gene products thereof selected from the group disclosed in any of Tables 2-6 and FIG. 11 and FIG. 15 indicates the sensitivity of the cells to the histone deacetylase inhibitor.

In certain preferred embodiments, an absence of expression of one or more genes or gene products thereof selected from the group disclosed in any of Tables 2-6 and FIG. 11 and FIG. 15 indicates resistance of the cell to the histone deacetylase inhibitor.

In certain preferred embodiments, the level of expression is the level of RNA in the cell.

In certain preferred embodiments, the level of expression is the level of protein encoded by the one or more genes.

In certain preferred embodiments, the cell is a tumor cell or a tumor cell line.

In certain preferred embodiments, the cell is in vitro.

In certain preferred embodiments, sensitivity of the cell indicates sensitivity of a tumor or tumor cell line to therapy with the histone deacetylase inhibitor.

In certain preferred embodiments, the cell is in vivo.

In certain preferred embodiments, sensitivity of the cell indicates sensitivity of a patient to therapy with the histone deacetylase inhibitor.

To develop transcriptional biomarkers of HDAC inhibitors, e.g. MGCD0103, in blood oriented samples, we first used microarray analysis to compare gene transcription in various samples, as described in Example 2. For example, Table 2 shows common genes whose transcription is upregulated by MGCD0103 in both human peripheral white cells ex vivo and in human colon HCT15 cells in vitro at 1 uM. Table 3 shows genes whose transcription is regulated by MGCD0103 in vivo in human H460 tumors in mice. Table 4 shows induction of proapoptotic proteins in human leukemic MV-4-11 cells in vitro by MGCD0103 at 1 uM. Table 5 shows time-dependent induction of gene transcription of antitumor excellular factors in an AML patient (Patient A) who has a clinical response and whose HDAC inhibition is 68% at day 8. Transcription of these genes was not induced (<=1 fold) in three other AML patients with an average HDAC inhibition <5% at day 8. Table 6. shows genes whose transcription is synergistically induced by Vidaza and MGCD0103 in an AML patient (H) with clinical response (CR) compared to an AML patient (I) with stable disease (SD).

This subset of genes is in no way intended to be limiting in nature as other genes including, but not limited to, those involved in cell cycle blocking, apoptosis and/or an anti-proliferation pathway are also expected to serve as biomarkers according to the present invention.

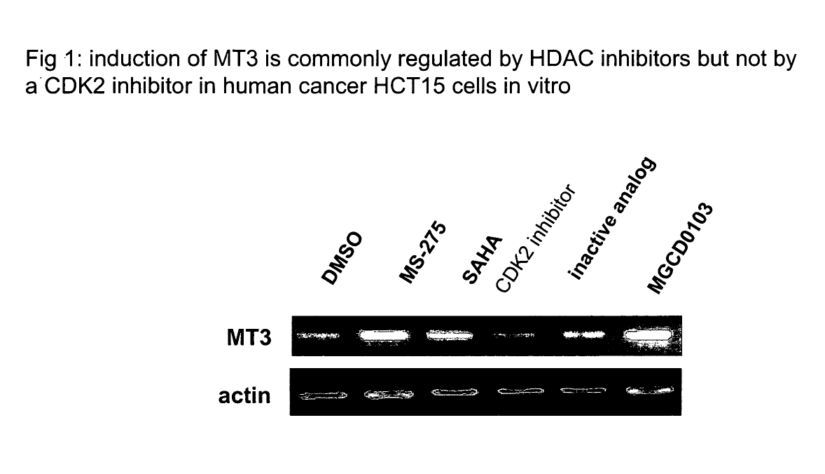

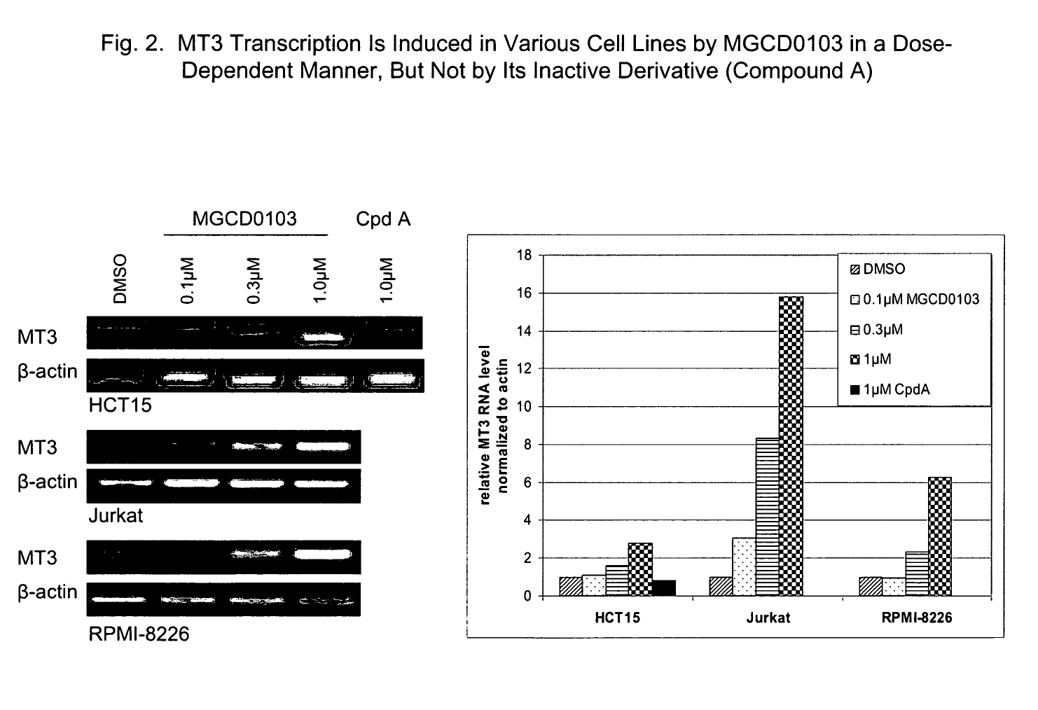

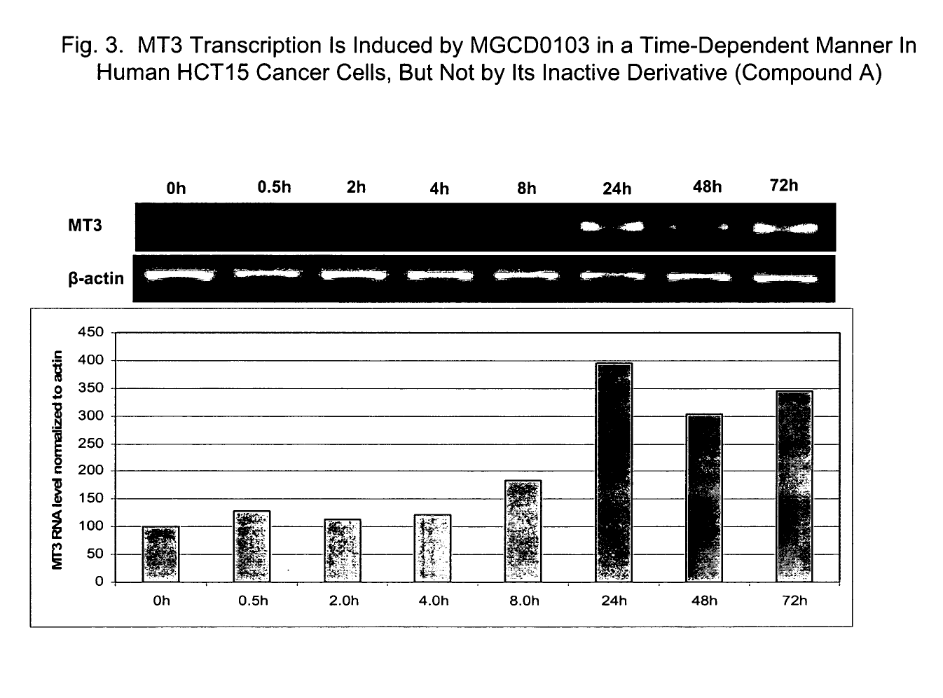

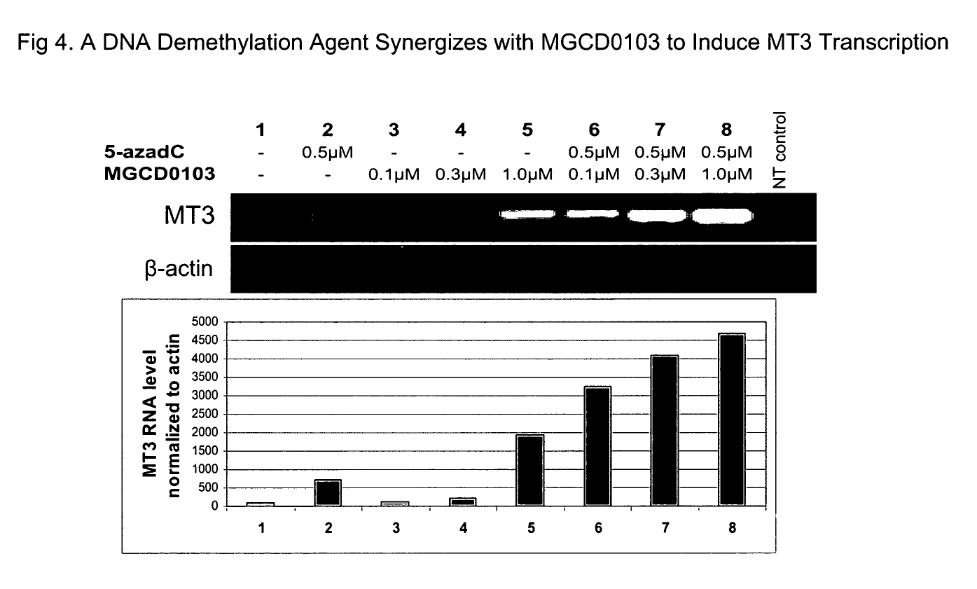

We then confirmed by RT-PCR that transcription of MT3 in human colon cancer HCT15 cells was induced in vitro by HDAC inhibitors MGCD0103, MS-275 and SAHA, but not by an inactive analog of MGCD0103 (Compound A) or a CDK inhibitor (FIG. 1) (See Table 1 for structures). Dose-dependent induction of MT3 transcription by MGCD0103 was observed in vitro in various human cancer cell lines from different tissue origins, including colon cancer HCT15 cells, Jurkat-T leukemic cells and RPMI-8226 myeloma cells (FIG. 2). Among them, MT3 transcription is also upregulated by MGCD0103, but not by its inactive analog, in HCT15 cells in a dose-dependent manner (FIG. 2). Time-dependent induction of MT3 transcription in HCT15 cells by MGCD0103 was observed in vitro (FIG. 3). Synergistic induction of MT3 transcription by MGCD0103 and a demethylating agent (5-aza-deoxyC) was demonstrated in human gastric carcinoma MKN45 cells in vitro (FIG. 4).

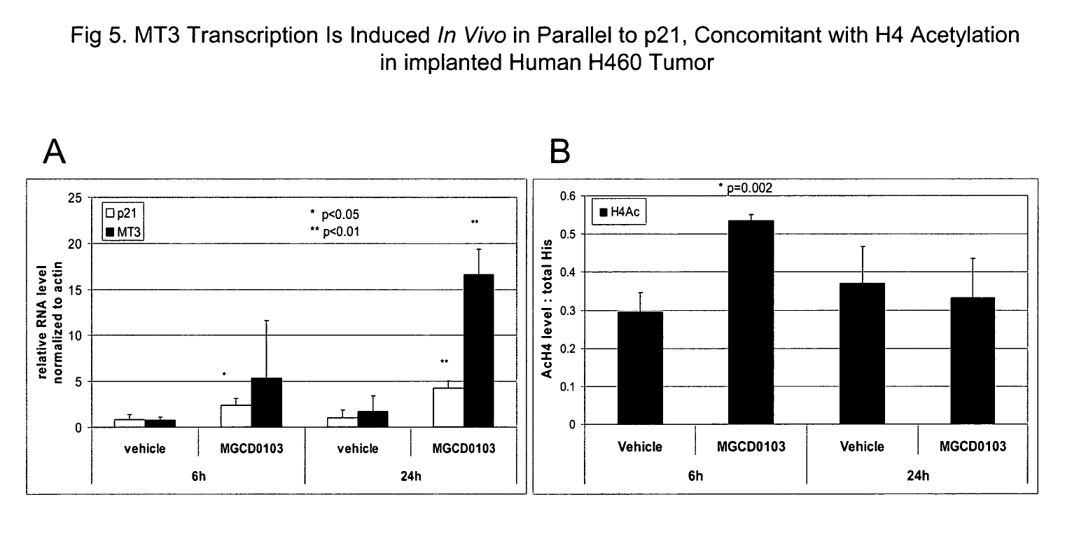

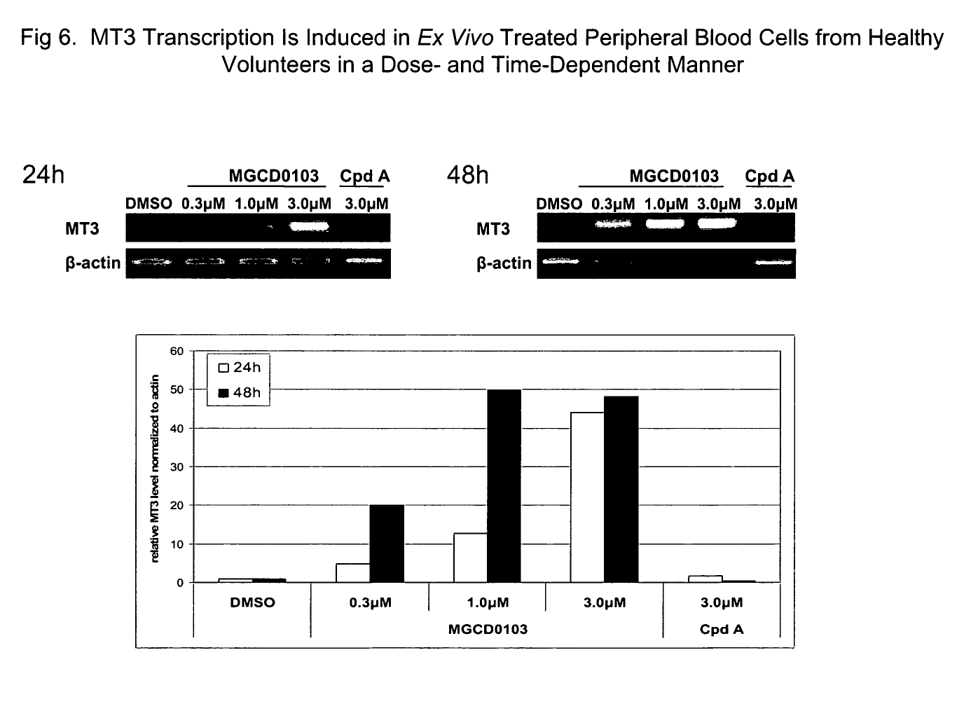

Next, we showed that induction of MT3 and p21 took place in implanted human NSCLC H460 tumors in vivo in nude mice (FIG. 5). FIG. 5, Panel A shows p21 and MT3 RNA levels quantified by conventional semi-quantitative PCR, normalized to β-actin. FIG. 5, Panel B shows H4Ac level detected by immunoblot analysis, normalized to total histones. H4 acetylation is detectable only after 6 h of treatment, but gene induction persists until at least 24 h. Induction of MT3 transcription occurred ex vivo in both a time-dependent and dose-dependent manner (FIG. 6).

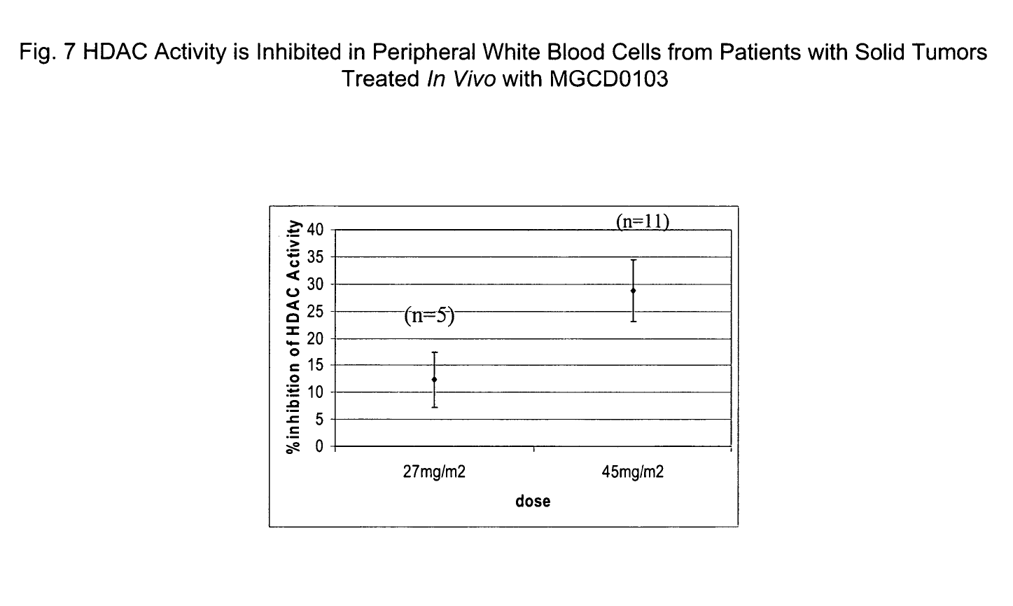

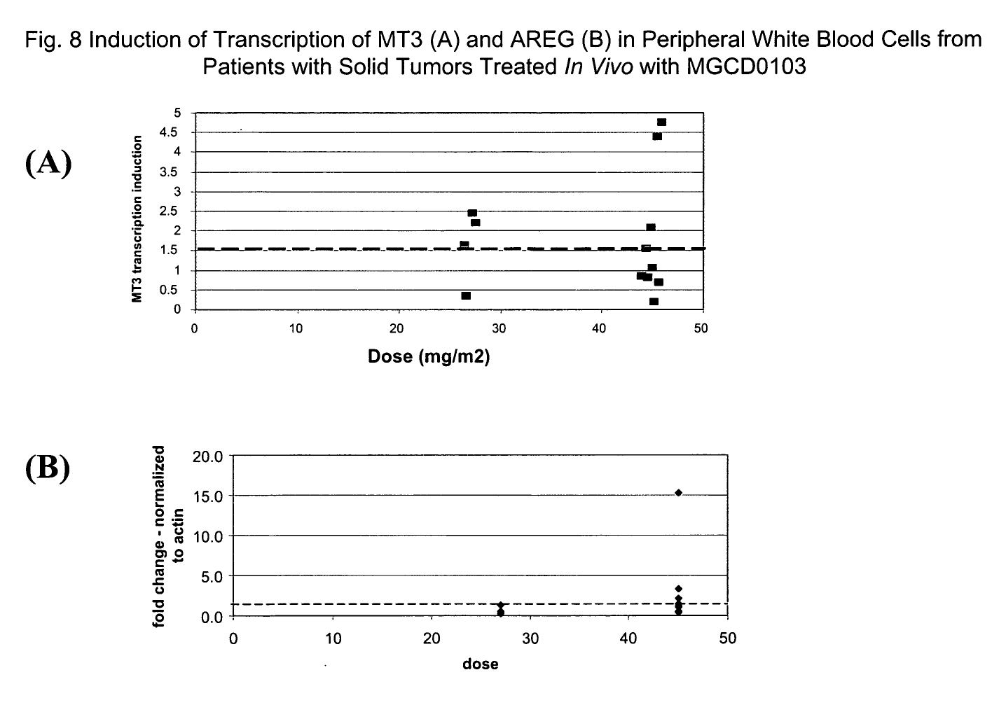

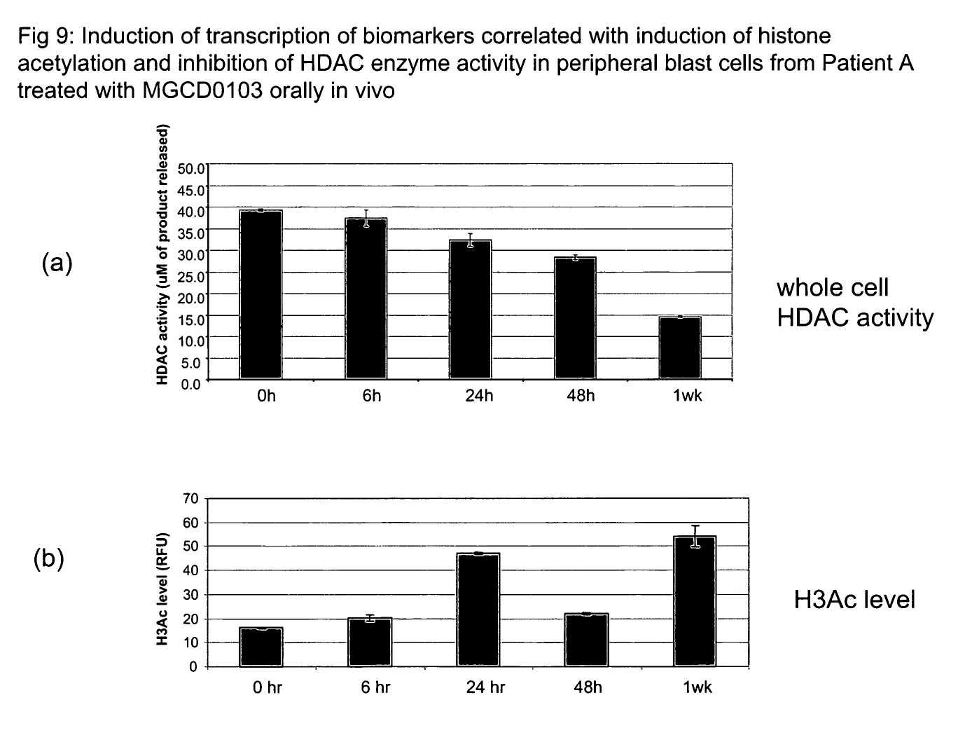

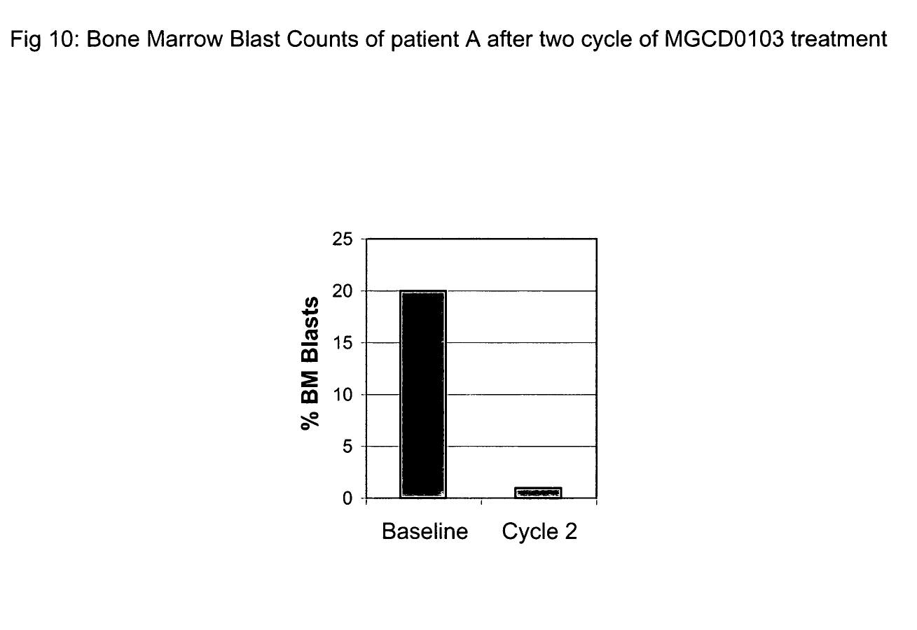

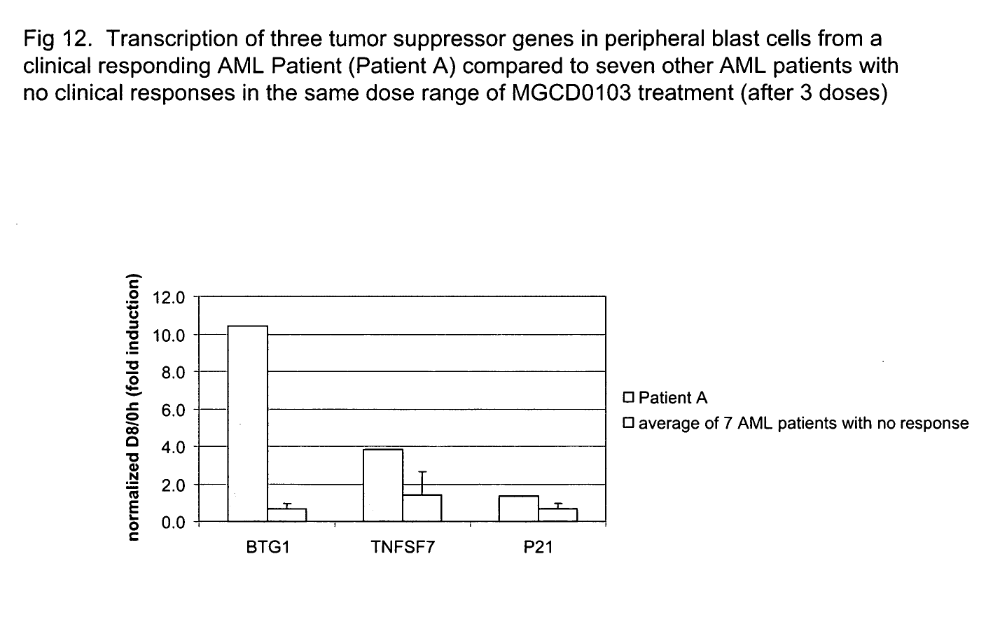

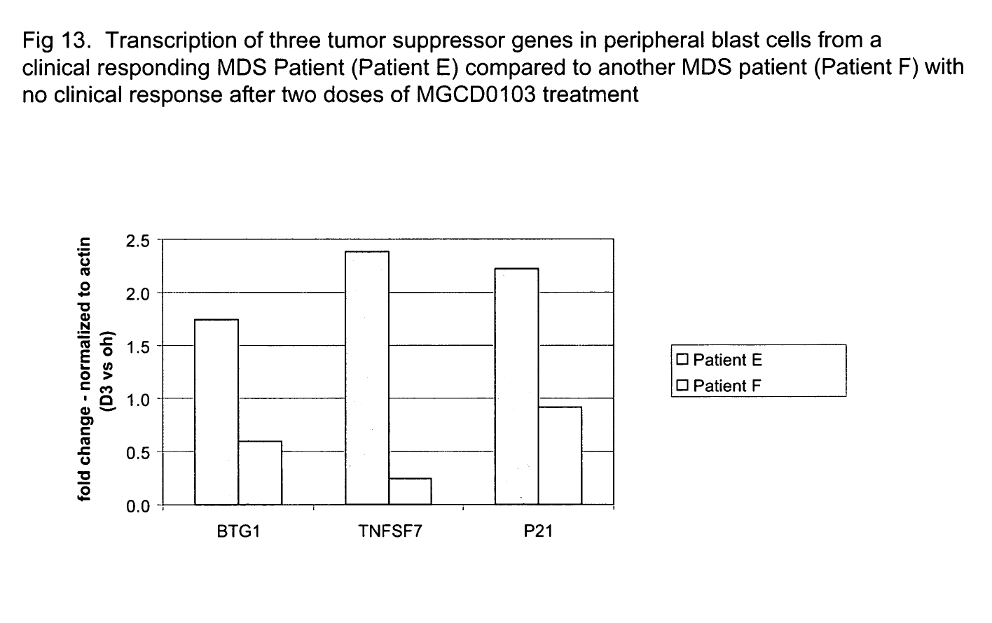

Then, dose-dependent HDAC inhibition was shown in vivo in patients with solid tumors who received treatment with MGCD0103 for one week (three doses per week) (FIG. 7). Also, induction of MT3 (A) and AREG (B) transcription in vivo was observed in patients with solid tumors who received treatment with MGCD0103 for one week (three doses per week) (FIG. 8). Time-dependent inhibition of HDAC activity and induction of histone H3 acetylation was seen in peripheral white cells from an AML patient (Patient A) who achieved a clinical response after MGCD0103 treatment in vivo (FIG. 9). In Patient A, 2 cycles of MGCD0103 treatment in vivo resulted in reduction of bone marrow blast count (FIG. 10). Induction of transcription of a subset of proapoptotic proteins was observed in peripheral blast cells (sample from D8 vs 0 h) from an AML patient with response (Patient A), in contrast to those from another three AML patients with no response (Patients B, C and D) in the same dose range, as determined by microarray expression analysis (FIG. 11). Induction of transcription of three tumor suppressor genes, BTG1, TNFSF9 and p21 in peripheral white cells from Patient A in vivo was determined by real time RT-PCR (FIG. 12). Induction of transcription of three tumor suppressor genes, BTG1, TNFSF9 and p21 was seen in peripheral white cells from a MDS patient with clinical response (Patient E), but not in a MDS patient without response (Patient F) in vivo as determined by real time RT-PCR (FIG. 13).

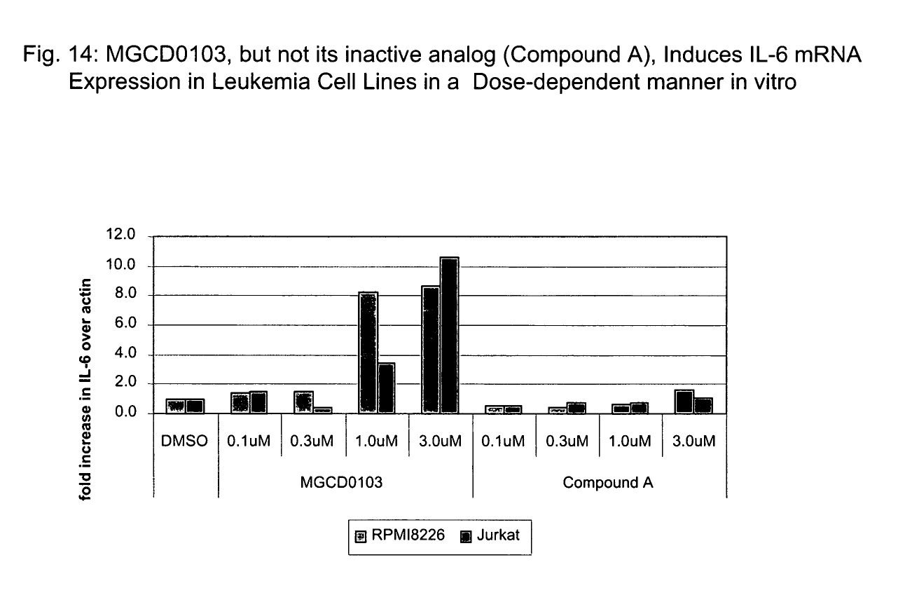

We next wanted to see whether readily identifiable cytokines could be used as surrogate markers for efficacy of HDAC inhibitors. First, dose-dependent induction of IL-6 transcription was shown in human leukemia RPMI8226 and Jurkat cells treated by MGCD0103 but not its inactive analog Compound A (FIG. 14). Then, induction of transcription of IL-6, IL-8, IL-1b, MIP1b cytokine/chemokines by MGCD0103 was shown in peripheral white cells ex vivo from a MDS patient (Patient J) and in vivo in an AML patient with response (Patient A) (FIG. 15). No such induction was observed in peripheral white cells from healthy volunteers ex vivo treated with MGCD0103 or two other AML patients without clinical response (Patient B, C) treated with MGCD0103 in vivo (FIG. 15). Induction of IL-6/IL-8/IL-1b/MIP1b by MGCD0103 ex vivo/in vivo in patients is specific as the expression level of other cytokine/chemokines is either decreased or remained unmodified (data not shown).

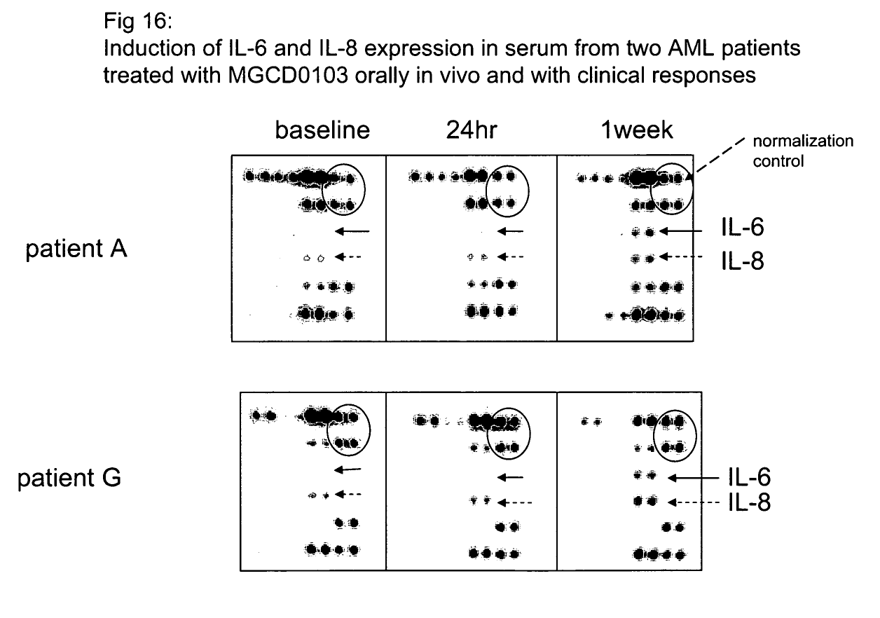

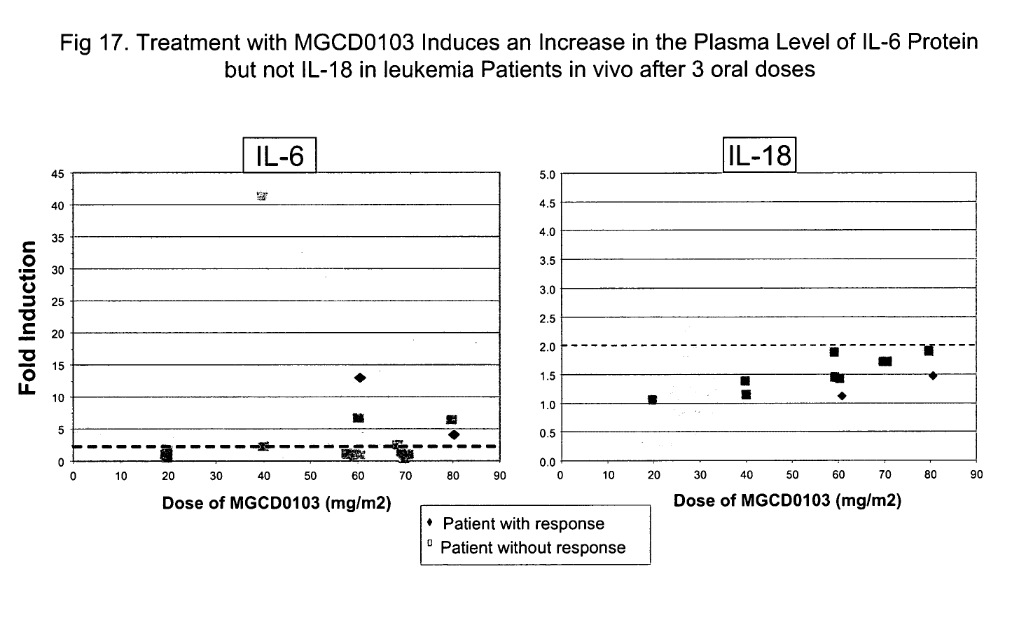

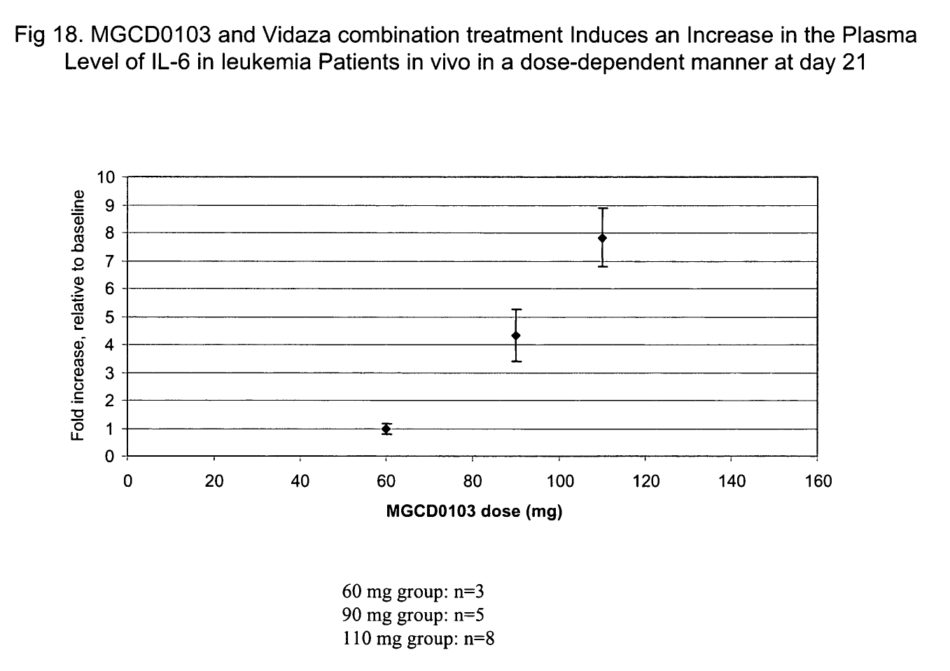

We next attempted to extend these findings to a clinical environment friendly format. This was first demonstrated by induction of IL-6 and IL-8 expression in plasma from two leukemia patients treated with MGCD0103 orally in vivo, as determined by cytokine antibody array (FIG. 16). Then, induction of plasma IL-6 from leukemia patients from treated with MGCD0103 at Day 8 was successfully analyzed by ELISA assays (FIG. 17). Finally, dose-dependent induction of IL-6 was demonstrated in plasma from AML/MDS patients by combination treatment of Vidaza and MGCD0103 (FIG. 18). Patient plasma samples at day 21 were analyzed by ELISA.

The following examples are provided to further illustrate certain particularly preferred embodiments of the invention and are not intended to limit the scope of the invention.

Chemicals

MGCD0103, its inactive analog, MS-275, SAHA and a CDK2 inhibitor were synthesized in house. The structures of MGCD0103, its inactive analog and the CDK2 inhibitor are shown in Table 1.

TABLE 1
Compounds described in application

| Structure | Compound |
|---|---|
| 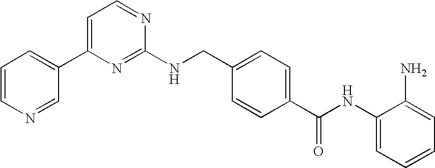 | MGCD0103 |
| 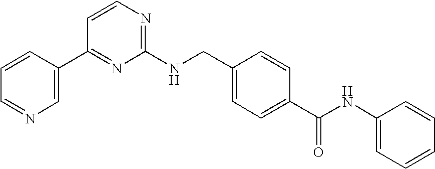 | inactive analog |
| 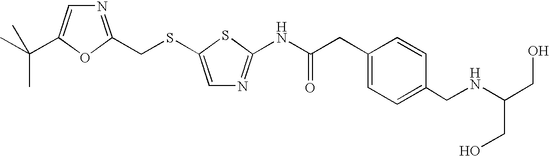 | CDK2 inhibitor |

All other chemicals were purchased from Sigma-Aldrich Canada Ltd., Oakville, Ontario.

### EXAMPLE 1

### Preparation of Cells for Analysis

Whole blood from either consenting healthy volunteers or consenting patients was centrifuged at 2500 rpm for 10 minutes at ambient temperature in a Sorvall RT-7 centrifuge (Mandel Scientific Co., Guelph, Ontario). Plasma was removed and buffy coat was collected. Five volumes of Erythrocyte Lysis Buffer (EL) (Qiagen Canada Inc., Mississauga, Ontario) were added into buffy coat. Buffy coat was incubated on ice for 20 minutes before it was centrifuged at 400 g for 10 minutes at 4° C. Supernatant was removed and buffy coat was washed twice with EL buffer and re-centrifuged. Buffy coat was resuspended in RPMI media and cells were counted with trypan blue exclusion. To isolate PBMCs, buffy coat cells were centrifuged over Lymphoprep (Axis-Shield, 1114544), and any remaining erythrocytes in the sample were lysed by treatment with EL lysis buffer (Qiagen, 79217). The cell pellets are washed and then re-suspended in RPMI containing 10% FBS.

Human cancer cell lines were from American Type Culture Collection (Manassas, Va.) and were all cultured following the vendor's instructions.

### EXAMPLE 2

### Microarray Gene Expression Analysis

RNA quality analysis was done using Agilent 2100 bioanalyzer and Agilent's RNA Labchip kits. RNAs were labeled with either Cy3 or Cy5 using Agilent's optimized labeling kits and hybridized to Human whole genome 44K Oligo Microarray (Agilent, Palo Alto, Calif.). Slides were scanned using DNA microarray scanner from

Agilent and the raw data was extracted using Agilent's image analysis tool (feature extraction software). Normalization and statistical analysis were performed using GeneSpring software. Biological analysis was performed using Biointerpreter software.

For Table 2, selected common genes whose transcription is upregulated (>=2.5 fold) both by MGCD0103 (at 1 uM) in human peripheral white blood cells ex vivo and human colon cancer cells in vitro were selected. Gene list was selected from a common list picking genes whose product is implicated in cell cycle arrest, apoptosis and anti-angiogenesis as well as expressed in excellular space.

TABLE 2
common genes whose transcription is upregulated by MGCD0103 in both human peripheral
white cells ex vivo and in human colon HCT15 cells in vitro at 1 uM

| | HCT15 | WBC | | | |
|---|---|---|---|---|---|
| Systematic | Fold Induction | | Common | Synonyms | Genbank |
| A_23_P114947 | 2.75 | 4.43 | regulator of G-protein signalling 2, 24 kDa | RGS2 | NM_002923 |
| A_24_P5856 | 2.96 | 3.32 | tumor necrosis factor (ligand) superfamily, member 9 | TNFSF9 | NM_003811 |
| A_23_P142506 | 3.10 | 2.90 | growth arrest and DNA-damage-inducible, beta | GADD45B | NM_015675 |
| A_23_P94754 | 4.11 | 4.43 | tumor necrosis factor (ligand) superfamily, member 15 | TNFSF15 | NM_005118 |
| A_24_P217520 | 4.14 | 10.48 | epiregulin | EREG | NM_001432 |
| A_23_P16523 | 4.18 | 18.75 | growth differentiation factor 15 | GDF15 | NM_004864 |
| A_23_P41344 | 4.62 | 12.77 | epiregulin | EREG | NM_001432 |
| A_23_P149019 | 5.13 | 2.69 | brain-specific angiogenesis inhibitor 2 | BAI2 | NM_001703 |
| A_23_P52336 | 5.70 | 2.66 | Unc-5 homolog B (C. elegans) | UNC5B | NM_170744 |
| A_23_P315273 | 7.65 | 5.16 | metallothionein 3 (growth inhibitory factor (neurotrophic)) | MT3 | NM_005954 |
| A_23_P151046 | 9.29 | 3.12 | killer cell lectin-like receptor subfamily C, member 1 | KLRC1 | NM_002259 |
| A_23_P259071 | 9.39 | 32.64 | amphiregulin (schwannoma-derived growth factor) | AREG | NM_001657 |

In Table 3, Selected genes whose transcription is upregulated (>=2.5 fold) MGCD0103 in vivo in implanted H460 tumors. Genes were selected by a common list by picking genes whose product is implicated in cell cycle arrest, apoptosis and anti-angiogenesis as well as expressed in excellular space.

TABLE 3
Selected genes whose transcription is regulated
by MGCD0103 in vivo in human H460 tumors in mice

| Fold | | | |
|---|---|---|---|
| induction | Common | Genbank | GeneSymbol |
| 3.4 | Chemokine (C-X-C motif) | NM_004887 | CXCL14 |
| | ligand 14 | | |
| 3.2 | immediate early response 3 | NM_003897 | IER3 |
| 3.1 | prominin 1 | NM_006017 | PROM1 |
| 3.1 | cyclin-dependent kinase | NM_000076 | CDKN1C |
| | inhibitor 1C (p57, Kip2) | | |
| 2.8 | superoxide dismutase 2, | AL050388; | SOD2 |
| | mitochondrial | | |
| 2.8 | SNAP25-interacting protein | NM_025248 | SNIP |
| 2.8 | tumor necrosis factor (TNF | NM_000594 | TNF |
| | superfamily, member 2) | | |
| 2.6 | Keratin, hair, acidic, 2 | NM_002278 | KRTHA2 |
| 2.2 | metallothionein 3 (growth | NM_005954 | MT3 |
| | inhibitory factor | | |
| | (neurotrophic)) | | |

In Table 4, genes whose transcription is upregulated (>=2.5 fold) in all three slides of a triplicate were picked. Gene list was further narrowed down by picking genes whose product is implicated in induction of apoptosis.

TABLE 4
Induction of proapoptotic proteins in human leukemic MV-4-11 cells in vitro by MGCD0103 at 1 uM

| | | Ratio vs control | | | | |
|---|---|---|---|---|---|---|
| | | | | | sample | |
| Gene | | Sample | sample | sample | 3 dye | |
| Symbol | Genbank | 1 | 2 | 3 | swap | |
| BMF | NM_001003940 | 6.0 | 7.4 | 6.7 | 7.1 | Homo sapiens Bcl2 modifying factor (BMF), transcript variant 1, |
| | | | | | | mRNA [NM_001003940] |
| CD40 | NM_001250 | 8.0 | 7.2 | 7.0 | 8.7 | Homo sapiens CD40 antigen (TNF receptor superfamily member 5) |
| | | | | | | (CD40), transcript variant 1, mRNA [NM_001250] |
| TNFSF14 | NM_003807 | 5.1 | 3.7 | 3.9 | 4.6 | Homo sapiens tumor necrosis factor (ligand) superfamily, member |
| | | | | | | 14 (TNFSF14), transcript variant 1, mRNA [NM_003807] |
| HIPK2 | NM_022740 | 4.1 | 6.6 | 4.6 | 4.2 | Homo sapiens homeodomain interacting protein kinase 2 (HIPK2), |
| | | | | | | mRNA [NM_022740] |
| CASP7 | NM_033339 | 5.0 | 7.2 | 5.2 | 5.8 | Homo sapiens caspase 7, apoptosis-related cysteine protease |
| | | | | | | (CASP7), transcript variant gamma, mRNA [NM_033339] |
| IL1B | NM_000576 | 9.6 | 11.2 | 8.5 | 11.4 | Homo sapiens interleukin 1, beta (IL1B), mRNA [NM_000576] |
| GPR65 | NM_003608 | 7.8 | 6.5 | 7.9 | 6.6 | Homo sapiens G protein-coupled receptor 65 (GPR65), mRNA |
| | | | | | | [NM_003608] |
| EIF2AK2 | NM_002759 | 17.9 | 21.1 | 18.1 | 26.7 | Homo sapiens eukaryotic translation initiation factor 2-alpha |
| | | | | | | kinase 2 (EIF2AK2), mRNA [NM_002759] |
| BNIP3L | NM_004331 | 4.9 | 3.2 | 4.9 | 3.4 | Homo sapiens BCL2/adenovirus E1B 19 kDa interacting protein |
| | | | | | | 3-like (BNIP3L), mRNA [NM_004331] |
| AHR | NM_001621 | 11.3 | 10.5 | 13.1 | 10.2 | Homo sapiens aryl hydrocarbon receptor (AHR), mRNA |
| | | | | | | [NM_001621] |
| PRKAR2B | NM_002736 | 10.8 | 7.2 | 11.1 | 7.6 | Homo sapiens protein kinase, cAMP-dependent, regulatory, type |
| | | | | | | II, beta (PRKAR2B), mRNA [NM_002736] |
| CDKN1A | NM_000389 | 4.4 | 4.0 | 4.6 | 3.9 | Homo sapiens cyclin-dependent kinase inhibitor 1A (p21, Cip1) |
| | | | | | | (CDKN1A), transcript variant 1, mRNA [NM_000389] |
| ADORA1 | NM_000674 | 3.6 | 5.4 | 5.1 | 5.4 | Homo sapiens adenosine A1 receptor (ADORA1), mRNA |
| | | | | | | [NM_000674] |
| DNASE2 | NM_001375 | 10.5 | 11.4 | 12.1 | 12.9 | Homo sapiens deoxyribonuclease II, lysosomal (DNASE2), mRNA |
| | | | | | | [NM_001375] |
| TNFRSF21 | NM_014452 | 4.1 | 6.4 | 4.7 | 5.2 | Homo sapiens tumor necrosis factor receptor superfamily, member |
| | | | | | | 21 (TNFRSF21), mRNA [NM_014452] |
| LY86 | NM_004271 | 4.5 | 3.6 | 3.8 | 4.3 | Homo sapiens lymphocyte antigen 86 (LY86), mRNA [NM_004271] |
| APOE | NM_000041 | 5.9 | 6.3 | 6.0 | 6.1 | Homo sapiens apolipoprotein E (APOE), mRNA [NM_000041] |
| TNFSF10 | NM_003810 | 7.4 | 7.2 | 10.1 | 5.6 | Homo sapiens tumor necrosis factor (ligand) superfamily, member |
| | | | | | | 10 (TNFSF10), mRNA [NM_003810] |
| AXUD1 | NM_033027 | 2.5 | 3.9 | 3.1 | 3.1 | Homo sapiens AXIN1 up-regulated 1 (AXUD1), mRNA [NM_033027] |
| IL18 | NM_001562 | 4.5 | 7.9 | 6.5 | 5.5 | Homo sapiens interleukin 18 (interferon-gamma-inducing factor) |
| | | | | | | (IL18), mRNA [NM_001562] |
| IL3RA | NM_002183 | 4.4 | 4.3 | 5.1 | 3.3 | Homo sapiens interleukin 3 receptor, alpha (low affinity) |
| | | | | | | (IL3RA), mRNA [NM_002183] |
| NALP1 | BC051787 | 7.3 | 7.3 | 7.4 | 6.4 | Homo sapiens NACHT, leucine rich repeat and PYD (pyrin domain) |
| | | | | | | containing 1, mRNA (cDNA clone MGC: 57544 IMAGE: 5756099), |
| | | | | | | complete cds. [BC051787] |
| MX1 | NM_002462 | 35.8 | 27.5 | 38.0 | 23.2 | Homo sapiens myxovirus (influenza virus) resistance 1, |
| | | | | | | interferon-inducible protein p78 (mouse) (MX1), |
| | | | | | | mRNA [NM_002462] |
| CLU | NM_203339 | 20.2 | 13.8 | 14.7 | 14.4 | Homo sapiens clusterin (complement lysis inhibitor, SP-40, 40, |
| | | | | | | sulfated glycoprotein 2, testosterone-repressed prostate |
| | | | | | | message 2, apolipoprotein J) (CLU), transcript variant 2, |
| | | | | | | mRNA [NM_203339] |
| PDE1B | NM_000924 | 4.3 | 3.4 | 4.8 | 3.6 | Homo sapiens phosphodiesterase 1B, calmodulin-dependent (PDE1B), |
| | | | | | | mRNA [NM_000924] |
| BNIP3L | NM_004331 | 2.6 | 3.5 | 2.9 | 3.4 | Homo sapiens BCL2/adenovirus E1B 19 kDa interacting protein |
| | | | | | | 3-like (BNIP3L), mRNA [NM_004331] |
| CASP5 | NM_004347 | 2.7 | 2.5 | 3.0 | 2.6 | Homo sapiens caspase 5, apoptosis-related cysteine protease |
| | | | | | | (CASP5), mRNA [NM_004347] |
| CAST | NM_173060 | 2.7 | 3.0 | 2.5 | 3.2 | Homo sapiens calpastatin (CAST), transcript variant 2, mRNA |
| | | | | | | [NM_173060] |
| CASP4 | NM_033306 | 2.7 | 2.8 | 2.6 | 2.9 | Homo sapiens caspase 4, apoptosis-related cysteine protease |
| | | | | | | (CASP4), transcript variant gamma, mRNA [NM_033306] |
| CASP4 | NM_033306 | 2.7 | 3.0 | 2.5 | 3.1 | Homo sapiens caspase 4, apoptosis-related cysteine protease |
| | | | | | | (CASP4), transcript variant gamma, mRNA [NM_033306] |
| CARD10 | NM_014550 | 2.7 | 4.4 | 3.0 | 3.9 | Homo sapiens caspase recruitment domain family, member 10 |
| | | | | | | (CARD10), mRNA [NM_014550] |
| TNFRSF25 | NM_148968 | 4.2 | 4.0 | 4.4 | 3.6 | Homo sapiens tumor necrosis factor receptor superfamily, |
| | | | | | | member 25 (TNFRSF25), transcript variant 5, mRNA [NM_148968] |
| PPP3CA | NM_000944 | 2.7 | 3.0 | 3.3 | 2.6 | Homo sapiens protein phosphatase 3 (formerly 2B), catalytic |
| | | | | | | subunit, alpha isoform (calcineurin A alpha) (PPP3CA), mRNA |
| | | | | | | [NM_000944] |
| MAP3K14 | NM_003954 | 2.6 | 2.8 | 2.8 | 2.6 | Homo sapiens mitogen-activated protein kinase kinase kinase 14 |
| | | | | | | (MAP3K14), mRNA [NM_003954] |
| NGFR | NM_002507 | 3.0 | 2.7 | 2.6 | 2.8 | Homo sapiens nerve growth factor receptor (TNFR superfamily, |
| | | | | | | member 16) (NGFR), mRNA [NM_002507] |

In Table 5, genes whose transcription is upregulated (>=2.5 fold) in patient A (responder) but not other three patients without response (<=1 fold) were picked. Gene list was further narrowed down by picking genes whose product is implicated in cell cycle arrest, apoptosis and anti-angiogenesis as well as expressed in excellular space.

TABLE 5
Time-Dependent Induction of Gene Transcription of Antitumor
Excellular Factors In An AML Patient (Patient A) Who Has A Clinical
Response and Whose HDAC Inhibition is 68% at day 8.

| Fold of Induction | | | | |
|---|---|---|---|---|
| 24 hr | day 8 | Genbank # | gene symbol | |
| 2.3 | 7.3 | NM_006273 | CCL7 | chemokine (C-C motif) |
| | | | | ligand 7 |
| 1.4 | 12.2 | NM_002984 | CCL4 | chemokine (C-C motif) |
| | | | | ligand 4 |
| 1.4 | 2.5 | NM_000619 | IFNG | interferon, gamma |
| 1.4 | 2.8 | NM_003246 | THBS1 | thrombospondin 1 |

Transcription of these genes is not induced (<=1 fold) in three other AML patients with an average HDAC inhibition <5% at day 8.

In Table 6, genes whose transcription is upregulated (>=2.5 fold) in patient H (responder) but not patient I (non-responder) and genes whose transcription is synergistically induced by MGCD0103 and azacitidine were picked. Gene list was further narrowed down by picking genes whose product is implicated in cell cycle arrest, apoptosis and anti-angiogenesis as well as expressed in excellular space.

TABLE 6
Genes whose transcription is synergistically induced by Vidaza and MGCD0103 in an AML patient
(H) with clinical response (CR) compared to an AML patient (I) with stable disease (SD)

| | Patient H: CR | | | Patient I (SD) | | | |
|---|---|---|---|---|---|---|---|
| | Fold Induction | | | Fold Induction | | | |
| Systematic | day 5 | day 6 | day 21 | day 21 | Common | Synonyms | Genbank |
| A_24_P142118 | 2.6 | 1.8 | 13.0 | 1.2 | thrombospondin 1 | THBS1 | NM_003246 |
| A_23_P206212 | 2.3 | 1.7 | 10.5 | 0.7 | thrombospondin 1 | THBS1 | NM_003246 |
| A_23_P165333 | 2.4 | 1.7 | 7.1 | 0.7 | bridging integrator 1 | BIN1 | NM_139346 |
| A_23_P134935 | | | 7.0 | 0.9 | dual specificity phosphatase 4 | DUSP4 | NM_001394 |
| A_23_P42065 | 1.5 | 1.2 | 6.1 | 1.2 | Tumor necrosis factor | TNFRSF21 | NM_014452 |
| | | | | | receptor superfamily, | | |
| | | | | | member 21 | | |
| A_23_P30666 | 1.7 | 1.5 | 5.4 | 1.3 | Tumor necrosis factor | TNFRSF21 | NM_014452 |
| | | | | | receptor superfamily, | | |
| | | | | | member 21 | | |
| A_23_P7144 | | | 5.0 | 0.9 | chemokine (C-X-C motif) | CXCL1 | NM_001511 |
| | | | | | ligand 1 (melanoma growth | | |
| | | | | | stimulating activity, alpha) | | |
| A_23_P208900 | 1.5 | 1.8 | 4.8 | 1.3 | sema domain, | SEMA6B | NM_032108 |
| | | | | | transmembrane domain | | |
| | | | | | (TM), and cytoplasmic | | |
| | | | | | domain, (semaphorin) 6B | | |
| A_23_P360777 | 0.4 | 1.3 | 4.5 | 1.1 | neuregulin 1 | NRG1 | NM_013957 |
| A_23_P126735 | 1.3 | 1.8 | 4.4 | 0.9 | interleukin 10 | IL10 | NM_000572 |
| A_24_P918740 | | 1.3 | 4.0 | 0.6 | adenomatosis polyposis coli | APC | NM_000038 |
| A_23_P68401 | 1.6 | 1.6 | 3.7 | 1.0 | catenin, beta like 1 | CTNNBL1 | NM_030877 |
| A_24_P364363 | 0.8 | 1.4 | 3.5 | 0.6 | Tumor necrosis factor | TNFRSF1A | NM_001065 |
| | | | | | receptor superfamily, | | |
| | | | | | member 1A | | |
| A_32_P102062 | 1.1 | 1.6 | 3.4 | 0.8 | forkhead box O3A | FOXO3A | NM_001455 |
| A_23_P33723 | 1.4 | 0.9 | 3.3 | 1.6 | CD163 antigen | CD163 | NM_004244 |
| A_24_P237036 | 2.7 | | 3.3 | 0.5 | tumor necrosis factor (ligand) | TNFSF14 | NM_003807 |
| | | | | | superfamily, member 14 | | |
| A_24_P70002 | 1.5 | 2.2 | 3.1 | 1.1 | LATS, large tumor | LATS2 | BC071572; |
| | | | | | suppressor, homolog 2 | | |
| | | | | | (Drosophila) | | |
| A_23_P136493 | 0.9 | 1.3 | 2.9 | 0.8 | neuregulin 1 | NRG1 | NM_013962 |
| A_23_P315815 | | 1.2 | 2.9 | 0.8 | neuregulin 1 | NRG1 | NM_013961 |
| A_23_P345575 | 1.0 | 1.4 | 2.7 | 0.9 | forkhead box O3A | FOXO3A | NM_001455 |

day 5: after 5 repeated doses of Vidaza
day 6: 24 hr after MGCD0103 treatment
day 21: 3 weeks after treatment

### EXAMPLE 3

### RT-PCR for p21, GADD45α, BTG1, BCL6, MT3 and Actin

RNA was extracted from 8×10⁶ cells using the Qiagen RNeasy Mini kit (cat#74106, Qiagen, Missisauga Ont.) following the manufacturer's instructions. RT reaction was performed using the Expand Reverse Transcriptase kit from Roche (Cat#1 785 834 Roche Applied Biosciences, Laval, Que) with 1 μg total RNA together with 1 μl oligo(dT) primer (cat#y01212, Invitrogen-Canada, Burlington, Ont). cDNA was synthesized using an Eppendorf Mastercycler gradient PCR apparatus (Brinkmann Instruments Canada LTD., Mississauga, Ontario), using a two cycle protocol. For the 1^{st} cycle reaction mixture (0.5 μl H₂O, 4 μl of 5× Buffer, 2 μl DTT, 2 ul of 10 mM dNTPs, 0.5 μl RNAsin (cat#N211B, Promega, Fisher Scientific, Whitby, Ont.) and 1 μg of the Expand Reverse Transcriptase (Cat#1785834, Roche, Laval, Quebec)) was incubated at 65° C. for 10 minutes and then set on ice. A second cycle was performed at 42° C. for 1 hour. The resulting cDNA products were kept at 4° C. until used.

One ul of the resulting cDNA was used for every amplification reaction with 18.9 ul H₂O, 1.25 ul of 10 mM dNTPs, 0.5 ul of each primer pair, 2.5 ul of 10× Buffer. The PCR reaction was carried out in an Eppendorf Mastercycler gradient. The sequences of the primers used for the amplification of selected biomarkers, as well as the details of the PCR reaction cycles are featured below. All buffers came from Expand Long Template PCR system (Roche, Laval, Quebec, Cat#11681842001). All primers were synthesized by Invitrogen (Invitrogen-Canada, Burlington, Ont.)

p21 (NM_000389)
LEFT PRIMER
5′-gacaccactggagggtgact-3′ start262 to 281
RIGHT PRIMER
5′-caggtccacatggtcttcct-3′ start433 to 414
PRODUCT SIZE: 172
PCR condition: Tm = 53.2° C., 34cycles, 10x
buffer 2
BTG1 (NM_001731)
LEFT PRIMER
5′-ctgttcaggcttctcccaag-3′ start588 to 607
RIGHT PRIMER
5′-tcgttctgcccaagagaagt-3′ start783 to 764
PRODUCT SIZE: 196
PCR condition: Tm = 50° C., 26cycles, 10x
buffer 2
MT3(NM_005954)
Nested PCR
1^{st} round PCR 20 cycles
LEFT PRIMER
5′-gacatggaccctgagacctg-3′ start234 to 253
RIGHT PRIMER
5′-gtcattcctccaaggtcagc-3′ start559 to 540
PRODUCT SIZE: 326
PCR condition: Tm = 52.3° C., 20cycles, 10x
buffer 3
2nd round PCR
LEFT PRIMER
5′-agacctgcccctgcccttct-3′ start247 to 266
RIGHT PRIMER
5′-ccacacggaggggtgccttc-3′ start463 to 444
PRODUCT SIZE: 216 bp
PCR condition: Tm = 52.3° C., 20cycles, 10x
buffer 3
Actin(X00351)
LEFT PRIMER
5′-acgaaactaccttcaactccatc-3′ start865 to 887
RIGHT PRIMER
5′-tggtctcaagtcagtgtacaggt-3′ start1738 to 1716
Product size 873 bp
PCR condition: Tm = 52.3° C., 17cycles. 10x
buffer 2

Analysis of transcripts was performed using STORM 860 (Amersham Biosciences, Baie d'Urfe, Qubec). Transcription of genes was normalized to transcription of actin, which was performed in the same RT-PCR reaction. The relative expression level of gense was normalized to baseline signals.

Real Time RT-PCR

Quantitative PCR with SYBR Green I detection with Mastercycler® ep realplex (Eppendorf) was performed using LightCycler® 480 SYBR Green I Master (Roche Diagnostics). SYBR Green I assays were performed with 600 nM primers. All other reaction conditions were as described by the manufacturer. Amplification conditions were 5 minutes of initial denaturation at 95° C., followed by 40 cycles of each 15 seconds at 95° C., 15 seconds at 63.4° C., 20 seconds at 68° C., a melting curve from 60° C. to 95° C. was recorded. Quantification was performed using Pfaffl method, a relative quantification method in real-time PCR (Nucleic Acids Research 2001 29:2002-2007). Primers were synthesized by Invitrogen (Invitrogen-Canada, Burlington, Ont.).

Primers for real-time PCR:

BTG1(NM_001731)
LEFT PRIMER
5′-tgcagaccttcagccagag-3′
RIGHT PRIMER
5′-gcttttctgggaaccagtga-3′
TNFSF7(NM_001252)
LEFT PRIMER
5′-ctgccgctcgagtcactt-3′
RIGHT PRIMER
5′-ccccctgccagtatagcc-3′
P21(NM_000389)
LEFT PRIMER
5′-ccaagaggaagccctaatcc-3′
RIGHT PRIMER
5′-aagatgtagagcgggccttt-3′
IL6(NM_000600)
LEFT PRIMER
5′-attctgcgcagctttaagga-3′
RIGHT PRIMER
5′-gaggtgcccatgctacattt-3′
β-ACTIN(X00351)
LEFT PRIMER
5′-ctcttccagccttccttcct-3′
RIGHT PRIMER
5′-agcactgtgttggcgtacag-3′

MT3 (NM_005954)
LEFT PRIMER
5′-ccctgcggagtgtgagaagt-3′
RIGHT PRIMER
5′-tgcttctgcctcagctgcct-3′
AREG (NM_001657)
LEFT PRIMER
5′-tggattggacctcaatgaca-3′
RIGHT PRIMER
5′-actgtggtccccagaaaatg-3′

### EXAMPLE 4

### In Vivo Treatment of Mice Using MGCD0103

Human non-small cell lung carcinoma NSCLC-H460 cells (2 million) were injected subcutaneously in the animal flank and allowed to form solid tumors. Tumor fragments were passaged in animals for a minimum of three times before their use. Tumor fragments (about 30 mg) were implanted subcutaneously through a small surgical incision under general anesthesia to Balb/cA female nude mice (6-8 weeks old). Recipient animals were treated with either vehicle (0.1 N HCl) or MGCD0103 (2HBr salt, in 0.1 N HCl) 100 mg/kg orally. Tumors were harvested after one dose of either 6 hour or 24 hours post administration of vehicle or MGCD0103. Each experimental group contained 3 animals. Tumor tissues were deposited in RNAlater (Qiagen, Missisauga Ontario) for total RNA extraction.

### EXAMPLE 5

### Administration of MGCD0103 in Leukemia Patients as Single Agent In Vivo

Human patients with either solid tumors or leukemia/MDS diseases were enrolled in phase I studies with consensus forms. MGCD0103 were dosed into patients orally every other day (day 1, day 3, day 5 and day 8). Blood samples were withdrawled by using the Vacutainer sodium-heparin blood collection tubes (Becton Dickinson Laboratories, Franklin Lakes, N.J.) and shipped to the test site within 24 hours on ice-pack. Baseline samples were drawn immediately prior to the first drug dose, while the 24 hour samples were drawn at 24 hours post the first drug dose. The 48 hours samples were drawn at 48 hours post the first dose. For the 1 week samples, blood was drawn 72 hours after day 5 dose (3 accumulated doses during week 1).

### EXAMPLE 6

### Administration of MGCD0103 in Combination with Azacitidine In Vivo in Leukemia Patients

In patients with advanced MDS, relapsed/refractory AML, and untreated elderly with AML, azacitidine was administered at its approved dose and schedule: 75 mg/m² SC daily for the first 7 days of the 28-day cycle. MGCD0103 was co-administered orally starting at Day 5, 3 times/week at escalating doses from 35 to 135 mg. Patients treated with doses ranging from 60 to 110 mg were analyzed in this study.

### EXAMPLE 7

### Fluorescence-Based Whole Cell HDAC Enzyme Assay

Freshly trypsinized cells or cells in suspension were dispensed in 96-well black Costar E1A/RIA plates (Corning Inc., Corning, N.Y.). We typically used 5×10⁴ to 2×10⁵ cells per well, and 8×10⁵ white blood (mouse or human) cells/per well. Small molecule substrate Boc-Lys(Ac)-AMC (Bachem Biosciences Inc., King of Prussia, Philadelphia) were added to cell suspension with the final concentration of 300 uM. Cells were placed in a 37° C. incubator with 5% CO₂ for 90 minutes (in the case of white blood cells, we incubated for 60 min). Fluorescence was read immediately before adding stop mixture to get a background. Reaction was stopped by adding a freshly prepared Fluor-de-Lys™ deleveloper (Biomol, Plymouth Meeting, Philadelphia) with 1 uM TSA (Biomol) in assay buffer (25 mM Tris-HCl pH8.0, 137 mM NaCl, 2.7 mM KCl, 1 mM MgCl2) plus 1% NP-40. Fluorescence was developed for 15 minutes at 37° C. and read in a fluorometer (SPECTRAMAX GeminiXS, Molecular Devices, Sunnyvale, Calif.) with an excitation wavelength at 360 nm, emission at 470 nm, and a cutoff of 435 nm.

### EXAMPLE 8

### Preparation of Nuclear Lysates

Human white peripheral cells were resuspended in 50 ul cold lysis buffer (10 mM Tris-HCl pH8.0, 1.5 mM MgCl₂, 5 mM KCl, 0.5% NP-40, 5 mM Na butyrate plus protease inhibitors) and incubated on ice for 10 min. Cells were centrifuged at 200 rpm in an IEC Micromax centrifuge (Fisher Scientific Ltd., Nepean, Ontario) at 4° C. for 10-15 min and nuclei collected. Nuclei were washed with 50 ul lysis buffer by centrifugation at 2000 rpm at 4° C. for 10-15 min. Nuclei were resuspended in 35 ul ice cold Nuclear Lysis buffer (50 mM HEPES pH7.5, 500 mM NaCl, 1% NP-40, 1 mM EDTA, 10% glycerol, 5 mM NaButyrate and protease inhibitors) and sonicated 10 seconds using a VirSonic 300 sonicator (VirTis, Gardiner, N.Y.). Lysed nuclei were centrifuged at 15000 rpm at 4° C. for 5 min and supernatant collected for ELISA.

### EXAMPLE 9

### Analysis of Histone Acetylation of Nuclear Extracts from White Blood Cells

Black plates were coated with 50 ul of diluted anti-Histone antibodies (H11-4, Chemicon, Temecula, Calif.) (1:1000 in TBS) and incubated at ambient temperature for 2 hours. Plates were washed twice with 50 ul of PBS and blocked with 1% BSA+0.1% TritonX-100 in PBS (50 ul) for 45 minutes. 5 ug nuclear extracts are incubated in the plate with 25 ul of rabbit anti-acetyl-H3 (1:1000 diluted in blocking buffer, from Upstate Biotech., Charlottesville, Va.) for 40 min and then plates were washed 3 times in blocking buffer. 50 ul of detection antibody (1:8000 dilution in blocking buffer, HRP-coupled goat anti-rabbit from Sigma-Aldrich Canada Ltd., Oakville, Ontario) were added and incubated at ambient temperatures for 45 minutes. Plates were washed in PBS twice and the HRP substrate Amplex-Red (Invitrogen Canada Inc., Burlinton, Ontario) was used according to the manufacturer's instructions. Fluoroscence development was allowed for 60 minutes in foil and plates were read on a fluorometer (Gemini XS, Molecular Devices, Sunnyvale, Calif.) at Excitation at 550 nm and emission at 610 nm with a cutoff of 590 nm (Auto PMT, 15 reads/well). Data were analyzed using Excel.

### EXAMPLE 10

### Cytokine Arrays

10 ml blood from patients were centrifuged at 2500 RPM for 10 min at 10° C. Plasma was separated and frozen until used. Plasma was thawed, spun again at 2500 RPM for 10 min at 10° C. The levels of cytokines in the plasma were determined using the TranSignal Human Cytokine Antibody Array 1.0 (Cat#MA6120, Panomics Inc. Redwood City, Calif.) following the manufacturer's instructions. Briefly, membranes were blocked using 1× blocking buffer for one hour. Following 2 brief washes, 1.5 ml of plasma was incubated for two hours, blots were washed and probed with provided secondary antibodies as suggested by the manufacturer. Blots were developed by autoradiography, scanned and quantitated using the Cyclone Software. Data was calculated, plotted using Excel and expressed as fold induction from Day 8 following treatment over baseline Day 0 samples.

### EXAMPLE 11

### Determination of IL-6 Protein Levels by ELISA

Blood from patients was centrifuged at 2500 RPM for 10 min at 10° C. Plasma was separated and frozen until used. Plasma was thawed, and spun again at 2500 RPM for 10 min at 10° C. The level of IL-6 was determined by ELISA (eBioscience, San Diego, Calif.), following the manufacturer's instructions. IL-6 concentration (pg/ml) in the samples was calculated from a standard curve generated by using standard IL-6 also provided in the kit. The range of detection is from 2-200 pg/ml for IL-6. All the data was calculated and plotted using Excel.

### EXAMPLE 12

### Determination of IL-18 Protein Levels by ELISA

Plasma from human blood was obtained as described above. The level of IL-18 was determined using an ELISA kit from R&D Systems, Inc. (Cat#7620, R&D Systems, Inc., Minneapolis, Minn.) and following the manufacturer's instructions. Briefly plasma was diluted 1:2 in Assay diluent and incubated for one hour on the precoated plate provided. Following five washes conjugate antibody was added for an additional hour followed by five washes again. Substrate solution was added to the wells and following addition of Stop solution, the absorbance in each well was read at 450 nm with the reference wavelength at 620 nm. The IL-18 concentration (pg/ml) in the samples was calculated from a standard curve generated by using standard IL-18 also provided in the kit. 1:2.5 serial dilutions ranging from 1000 pg/ml 25.6 pg/ml were used to generate this standard curve.

1. A method for assessing the efficacy of a histone deacetylase inhibitor in a mammal comprising obtaining peripheral blood cells from a mammal that has not been treated with the histone deacetylase inhibitor; determining a level of expression in the peripheral blood cells of a set of at least one or more genes or gene products thereof selected from the group consisting of a cell cycle blocking gene, a cell cycle blocking gene product, a pro-apoptosis gene, a pro-apoptosis gene product, a non-apoptotic cell death gene, a non-apoptotic cell death gene product, an anti-proliferation gene, an anti-proliferation gene product, an anti-angiogenesis gene and an anti-angiogenesis gene product, a differentiation induction gene, a differentiation induction gene product, a gene encoding antitumor soluble factors, an antitumor soluble factor, a gene encoding transcriptional factor, a transcriptional factor, a gene encoding soluble factor, a soluble factor; treating the mammal with the histone deacetylase inhibitor; obtaining peripheral blood cells from the mammal treated with the histone deacetylase inhibitor; determining the level of expression in the peripheral blood cells from the mammal treated with the histone deacetylase inhibitor of the same set of at least one or more genes; and comparing the level of expression of the set of the at least one or more genes from the peripheral blood cells of the mammal that has not been treated with the histone deacetylase inhibitor against the level of expression of the set of at least one or more genes from the peripheral blood cells of the mammal after it has been treated with the histone deacetylase inhibitor, wherein increased expression of the set of at least one or more genes from the peripheral blood cells of the mammal after it has been treated with the histone deacetylase inhibitor relative to the level of expression of the set of the at least one or more genes from the peripheral blood cells of the mammal that has not been treated with the histone deacetylase inhibitor is indicative of efficacy of the histone deacetylase inhibitor in the mammal.

2. The method according to claim 1 wherein the genes or gene products thereof is selected from the group consisting of FOXO1A, IER3, UNC5B, GADD45B, RGS2, KLF4, TNFSF9, TNFSF15, PDCD1, KLRC1, KLRC4, YPEL4, CDKN1A (P21), GADD45b, BTG1 and MT3, EREG, GDF15, BAI2, AREG, CXCL14, PROM1, CDKN1C, SOD2, SNIP, TNF, KRTHA2, BMF, CD40, TNFSF14, HIPK2, CASP7, IL1B, GPR65, EIF2AK2, BNIP3L, AHR, PRKAR2B, ADORA1, DNASE2, TNFRSF21, LY86, APOE, TNFSF10, AXUD1, IL3RA, NALP1, MX1, CLU, PDE1B, CASP5, CAST, CASP4, TNFRSF25, PPP3CA, MAP3K14, NGFR, CCL7, CCL4 (MIP1b), IFNG, THBS1, BIN1, DUSP4, CXCL1, SEMA6B, NRG1, IL10, APC, CTNNBL1, TNFRSF1A, FOXO3A, CD163, TNFSF14, LASTS2, NRG1, RIPK1, CLC, TNFSF7, CASP8, ELMO2, TP53BP2, AD7C-NTP, CYCS, TRAF4, CIAS1, INHBA, PHLDA2, BCL2L11, IL-6, IL-8

3. The method according to claim 1 wherein the set of one or more genes comprises MT3, TNFSF7, BTG1, IL-6, IL-8, IL1b, CCL4, CCL7, IFNG, THBS1, BIN1, DUSP4, TNFRSF21, CXCL1, SEMA6b, NRG1, IL10, APC, CTNNBL1, TNFRSF1a, FOXO3a, CD163, TNFSF14, LAST2, CXCL14, IER3, PROM1, CDKN1c, SOD2, SNIP, TNF, KRTHA2.

4. The method according to claim 1 wherein the set of one or more genes comprises MT3, TNFSF7, BTG1, IL-6, IL-8, IL1b, CCL4, CCL7, IFNG, THBS1, TNFRSF21, CXCL1, NRG1, IL10, APC, TNFRSF1a, FOXO3a, BMF, ELMO2, BCL2L11.

5. A method for assessing the efficacy of a histone deacetylase inhibitor in a mammal comprising obtaining serum from a mammal that has not been treated with the histone deacetylase inhibitor, determining a level of a set of at least one or more circulating serum proteins in the serum from the mammal, treating the mammal with the histone deacetylase inhibitor, obtaining serum from the mammal treated with the histone deacetylase inhibitor, determining the level of the same set of at least one or more proteins in the serum from the mammal treated with the histone deacetylase inhibitor, and comparing the level of the set of at least one or more proteins in the serum from the mammal that has not been treated with the histone deacetylase inhibitor against the level of the set of at least one or more proteins in the serum from the mammal after it has been treated with the histone deacetylase inhibitor, wherein increased levels of the set of at least one or more proteins in the serum from the mammal after it has been treated with the histone deacetylase inhibitor relative to the level of the set of at least one or more proteins in the serum from the mammal that has not been treated with the histone deacetylase inhibitor is indicative of efficacy of the histone deacetylase inhibitor in the mammal.

6. The method according to claim 5, wherein the circulating serum protein is selected from the group consisting of a cytokine, a chemokine, a soluble receptor, a hormone and an antibody.

7. The method according to claim 5, wherein the circulating serum protein is selected from the group consisting of TNFSF9, TNFSF15, EREG, AREG, CXCL14, TNF, TNFSF14, IL1B, CCL7, CCL4 (MIP1b), IFNG, THBS1, CXCL1, IL10, NRG1, TNFSF7, IL-6, IL-8.

8. The use of a gene or gene product thereof identified according to claim 1 as a biomarker to predict a patient response to histone deacetylase inhibitor treatment.

9. A method for assessing efficacy of an HDAC inhibitor in a patient comprising obtaining a first sample of cells from the patient, treating the patient with the HDAC inhibitor, obtaining a second sample of cells from the patient, assessing the level of expression of one or more genes or gene products thereof from the group consisting of the genes disclosed in Tables 2-7 in the first sample of cells and in the second sample of cells, and comparing the level of expression of the one or more genes or gene products thereof in the first sample of cells with the level of expression of the one or more genes or gene products thereof in the second sample of cells, wherein the HDAC inhibitor is efficacious if the level of expression of the one or more genes or gene products thereof in the second sample of cells is greater than the level of expression of the one or more genes or gene products thereof in the first sample of cells.

10. The method according to claim 9, wherein the cells are blast cells.

11. The method according to claim 9, wherein the cells are peripheral blood cells.

12. The method according to claim 9, wherein the cells are tumor cells.

13. The method according to claim 10, wherein the cells are cells from skin biopsy.

14. The method according to claim 10, wherein the cells are cells from buccal swipe.

15. The method according to claim 9, wherein the level of expression of the on or more genes or gene products thereof in the second sample of cells is at least 2.5-fold greater than the level of expression of the one or more genes or gene products thereof in the first sample of cells.

16. The method of claim 9, wherein the level of expression is the level of RNA.

17. The method of claim 9, wherein the level of expression is the level of protein encoded by the one or more genes.

18. The method according to claim 12, wherein the one or more genes is selected from the group consisting of FOXO1A, IER3, UNC5B, GADD45β, RGS2, KLF4, IL-18, TNFSF9, TNFSF15, PDCD1, KLRC1, KLRC4, YPEL4, CDKN1A (P21), GADD45a, GADD45b, BTG1 and MT3, EREG, GDF15, BAI2, AREG, CXCL14, PROM1, CDKN1C, SOD2, SNIP, TNF, KRTHA2, BMF, CD40, TNFSF14, HIPK2, CASP7, IL1B, GPR65, EIF2AK2, BNIP3L, AHR, PRKAR2B, ADORA1, DNASE2, TNFRSF21, LY86, APOE, TNFSF10, AXUD1, IL3RA, NALP1, MX1, CLU, PDE1B, CASP5, CAST, CASP4, TNFRSF25, PPP3CA, MAP3K14, NGFR, CCL7, CCL4 (MIP1b), IFNG, THBS1, BIN1, DUSP4, CXCL1, SEMA6B, NRG1, IL10, APC, CTNNBL1, TNFRSF1A, FOXO3A, CD163, TNFSF14, LASTS2, NRG1, RIPK1, CLC, TNFSF7, CASP8, ELMO2, TP53BP2, AD7C-NTP, CYCS, TRAF4, CIAS1, INHBA, PHLDA2, BCL2L11, IL-6, IL-8.

19. The method according to claim 12 wherein the one or more genes comprises MT3, TNFSF7, BTG1, IL-6, IL-8, IL1b, CCL4, CCL7, IFNG, THBS1, BIN1, DUSP4, TNFRSF21, CXCL1, SEMA6b, NRG1, IL10, APC, CTNNBL1, TNFRSF1a, FOXO3a, CD163, TNFSF14, LAST2, CXCL14, IER3, PROM1, CDKN1c, SOD2, SNIP, TNF, KRTHA.

21. The method according to claim 12, wherein the one or more genes or gene product thereof is selected from the group consisting of MT3, TNFSF7, BTG1, IL-6, IL-8, IL1b, CCL4, CCL7, IFNG, THBS1, TNFRSF21, CXCL1, NRG1, IL10, APC, TNFRSF1a, FOXO3a, BMF, ELMO2, BCL2L11.

22. A method for screening a compound for HDAC inhibitory activity, comprising:
a) administering a compound to cells to obtain treated cells;
b) assaying for expression levels of a set of at least one or more genes selected from the group consisting of those disclosed in any of Tables 2-6, FIG. 11 and FIG. 15, in the treated cells and in control cells to which no compound has been administered; and
c) comparing the expression levels between the treated cells and the control cells wherein a difference in the expression levels between the treated cells and control levels indicates whether the compound possesses HDAC inhibitor activity.

23. The method of claim 22, wherein the expression levels is the level of RNA.

24. The method of claim 22, wherein the expression level is the level of protein encoded by the one or more genes.

25. The method according to claim 22, wherein the cells are selected from the group consisting of a blast cell, a blood cell, a tumor cell line and a tumor cell.

26. The method of claim 22, wherein the cells are in vivo.

27. The method of claim 22, wherein the cells are in vitro.
